(19) United States
(12) Patent Application Publication
(10) Pub. No.: US 2004/0086901 A1
(43) Pub. Date: May 6, 2004

(54) Database for analyzing gene function and method of analyzing gene function by dspa

(76) Inventor: Takaki Hiwasa, Chiba (JP)

Correspondence Address: WENDEROTH, LIND & PONACK, L.L.P., 2033 K STREET N. W., SUITE 800, WASHINGTON, DC, 20006-1021, US

(21) Appl. No.: 10/450,118

(22) PCT Filed: Dec. 11, 2001
(86) PCT No.: PCT/JP01/10838

(30) Foreign Application Priority Data
Dec. 12, 2000 (JP) 2000-378047
Dec. 10, 2001 (JP) 2001-375565

(51) Int. Cl. C12Q 1/68; G06F 19/00; G01N 33/48; G01N 33/50

(52) U.S. Cl. 435/6; 702/20

(57) ABSTRACT

A method of constructing a gene function database comprising measuring the cell viabilities, against a plural number of drugs at various concentrations, of transformed eukaryotic cells overexpressing a plural number of function-known genes and parental cell line thereof, calculating the ratios of IC₄₀ values of the transformed cells to the IC₄₀ values of the parental cell line from the viabilities, calculating the logarithmic values of the ratios, and calculating the correlation coefficients among the known genes based on these logarithmic values; the database constructed by such a method; and a method of analyzing gene function of unknown genes is elucidated from the correlation coefficients of the function-unknown gene to each of the known genes in the database.

### TECHNICAL FIELD

Method of this application relates to analysis of unknown gene function, to a gene function database used for the analysis and to a method constructing the database. More particularly, the invention of this application relates to a novel method for analyzing function of function-unknown gene which is useful as a genetic material for the pharmacogenomics and for the manufacture of various useful proteins by means of genetic engineering and to a gene function database used for the analysis as well as a method for constructing the database.

### BACKGROUND ART

As a result of the human genome project, all of human gene sequences will be soon elucidated. It is predicted that, in near future, causative genes for all genetic diseases will be made clear. Identification of causative genes for diseases is expected to greatly contribute in correct and simple diagnosis of diseases or in effective preventive treatment and therapy.

However, although many causative genes have been identified already, the greater part thereof has not yet been applied for the development of therapeutic drugs or others. That is because functions of the causative genes (functions of expression products) have not been elucidated yet. For example, even when the relevancy of the causative gene with pathology is made clear using knockout mice, etc., the action mechanism of a genetic product during that process is ambiguous and, therefore, it is not possible to search a compound (a lead compound) affecting a gene product (a target protein) and to develop the drug using such a compound.

Until now, analysis of gene function is greatly dependent upon discretion of researchers, and a lot of labor and expense have been paid for analysis of one gene function. It is predicted that, in future, identification of a gene product relating to an expression product of the causative gene for disease among huge numbers of genetic products will become more and more difficult, and there has been a strong demand for development of a new method for analysis of gene function.

The invention of this application has been achieved under the above-mentioned circumstances, and objects of the invention are to provide a novel method for a simple and correct analysis of function of function-unknown genes, a database for analyzing gene function used for the analysis method and a method of constructing the database.

### DISCLOSURE OF INVENTION

As an invention for solving the above-mentioned objects, this application provides a method for construction of a gene function database, which comprises:

(a) ensuring the viability against a plural number of drugs (D₁, D₂, D₃, . . . Dₙ) at various concentrations, of transformed eukaryotic cells overexpressing a plural number of function-known genes (g₁, g₂, g₃, . . . gₙ) and their parental cell lines;

(b) calculating the ratio of the concentration value of the drug to inhibit the viability of the transformed cell to an extent of 40% (IC₄₀ value) to the IC₄₀ value of the parental cell line;

(c) calculating the logarithmic values of the ratios of the above (b) for the known genes (g₁, g₂, g₃, . . . gₙ); and

(d) calculating the correlation coefficients among the known genes (g₁, g₂, g₃, . . . gₙ) for the logarithmic values of the above (c).

In the method for constructing the database, it is a preferred embodiment that “n” of the known genes (gₙ) is 50 or more, and that “n” of the drugs (Dₙ) is 40 or more.

This application further provides a gene function database, which is constructed by the above constructing method.

This application furthermore provides a method of analyzing functions of a function-unknown gene (gₓ) on the basis of DSPA (Drug Sensitivity Pattern Analysis) using the gene function database set forth above, which comprises:

(i) measuring the IC₄₀ value for each drug from the viability at various concentrations of a plural number of drugs (D₁, D₂, D₃, . . . Dₙ) for transformed eukaryotic cells overexpressing the unknown gene (gₓ),

(ii) calculating the correlation coefficients between the unknown gene (gₓ) and known genes (g₁, g₂, g₃, . . . gₙ) from the IC₄₀ values of the above (i) by the same method as in the calculation of the correlation coefficient among the known genes (g₁, g₂, g₃, . . . gₙ) of the gene function database, and

(iii) determining that the function of the known gene showing a significant correlation coefficient to the unknown gene (gₓ) is related to the function of the unknown gene (gₓ).

This application still further provides a method for constructing the database according to claim 1, wherein the data of the unknown gene (gₓ) whose function is determined by the method of analyzing functions is added to database as a data of the function-known gene, and still furthermore provides a gene function database constructed by the constructing method set forth above.

### BRIEF DESCRIPTION OF DRAWINGS

FIG. 1 is examples of selection of high-expression cells by Western blotting method.

FIG. 2 is an example of calculating the ratio of IC₄₀ values from a curve showing the dependency of concentration of drug on viability. The range of an arrow in the drawing shows log 0.23=-0.64.

FIG. 3 is another example for calculating the ratio of IC₄₀ values from a curve showing the dependency of concentration of drug on viable rate.

FIG. 4 shows graphs where drug-sensitivity ratios of genes having high functional relationship (IkB-SR and IKK-DN) and genes having low functional relationship (IkB-SR and p16) are subjected to a logarithmic plotting.

### BEST MODE FOR CARRYING OUT THE INVENTION

The gene function database of this application is constructed by the following steps (a) to (d).

Step (a):

There are measured the viabilities, against a plural number of drugs (D₁, D₂, D₃, . . . Dₙ) at various concentrations, of transformed eukaryotic cells overexpressing a plural number of function-known genes (g₁, g₂, g₃, . . . gₙ) and their parental cell lines.

The function-known genes are those where functions of the expression products thereof have been known already and, with regard to their numbers, not less than 50 or, preferably, not less than 100 genes are used. Full-length cDNA of each of those genes is integrated into an expression vector for eukaryotic cells and the recombinant vector is transfected into eukaryotic cells. With regard to the expression vector, known vectors such as pRc-CMV, pcDNA3 and pMSG may be appropriately used. With regard to the eukaryotic cells, there may be exemplified cell lines such as mouse fibroblast cell NIH3T3 and Ha-ras-NIH3T3 although they are non-limitative. Transfectants may be selected using an appropriate selecting drug depending upon the type of the drug-resistant gene of the vector. All of cell lines into which each of the genes (g₁, g₂, g₃, . . . gₙ) is introduced are checked for gene expression by Western blotting method or Northern blotting method, and the most highly expressed cell line is selected.

Then, with regard to those gene-introduced cells and the parental cell line thereof, their viabilities to a plural number of drugs at various concentrations are measured. The drugs are physiologically active substances (such as cytokines) or drugs which have been known to affect the viabilities of the parental cell line, and they are other than the object drugs for the drug-resistant gene owned by the vector used for the introduction of the gene. Forty kinds or more of drugs are used.

Cell viabilities can be measured by various known methods, and MTT method is preferred. The MTT method is a method where coloration of formazan which is a metabolite of MTT dye (tetrazolium salt 3-(4,5-dimethylthiasol-2-yl)-2,5-diphenyltetralin bromide) by mitochondrial succinic dehydrogenase of growing cells is measured (J. Immunol. Methods 65: 55-63, 1983; J. Immunol. Methods 116: 151-158, 1989) and, because of its quickness and precision, it has been used as a method for measuring the cell growth.

Step (b):

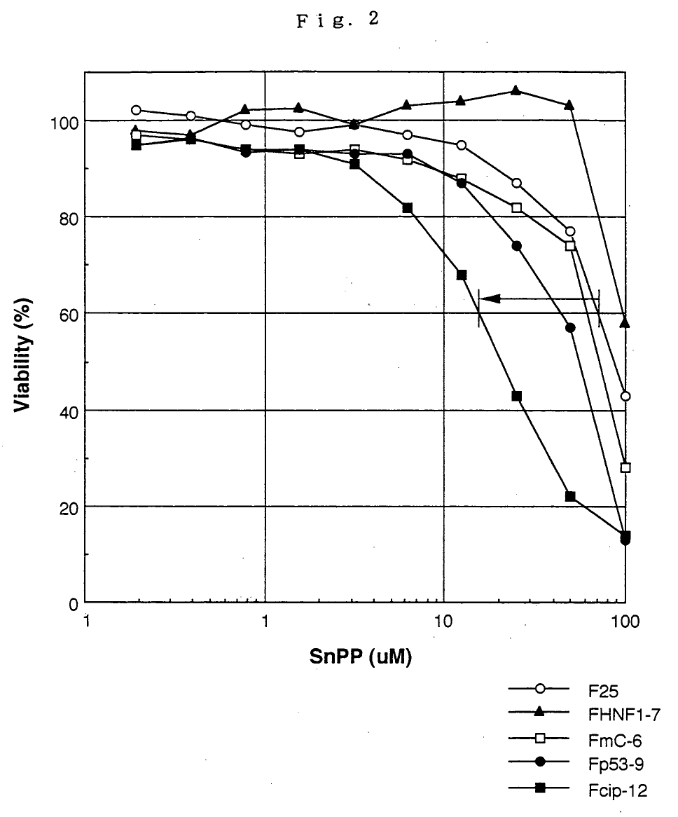

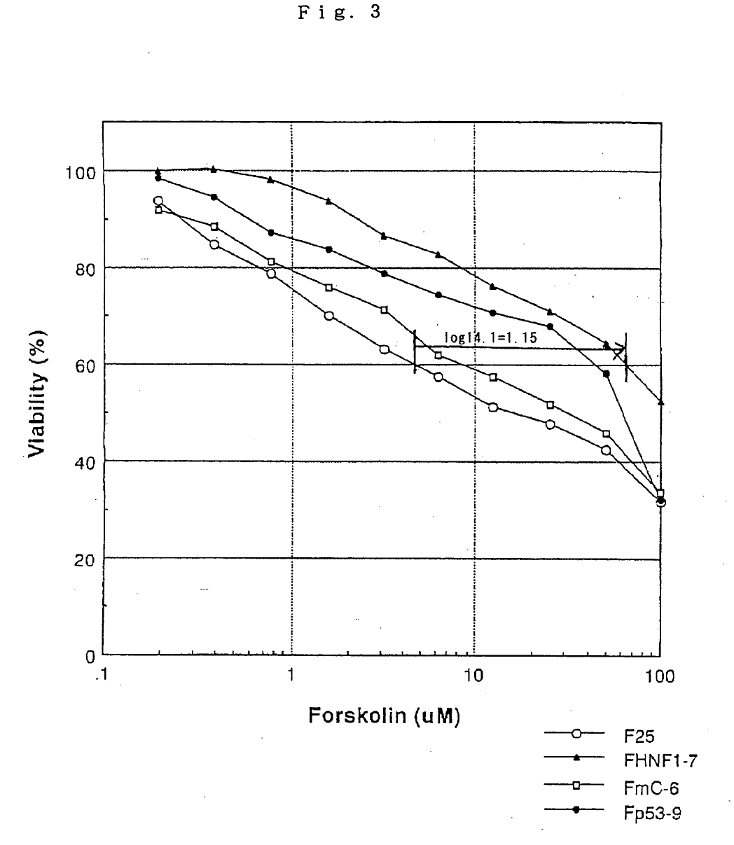

There are calculated the ratios of the concentration value of the drug to inhibit the viabilities of the transformed cell to an extent of 40% (IC₄₀ value) to the IC₄₀ value of the parental cell line. Thus, for example, ratio of the IC₄₀ values can be calculated from the concentration-dependent curve on the viability as shown in FIG. 2 and FIG. 3 prepared in the Examples mentioned later.

Step (c):

With regard to all of the known genes (g₁, g₂, g₃, . . . gₙ), there are calculated the logarithmic values of the ratios calculated in the above step (b). The logarithmic values are inputted, for example, into a list (Table 2) prepared in the Examples mentioned later.

Step (d):

There are calculated the correlation coefficients among the known genes (g₁, g₂, g₃, . . . gₙ) for the logarithmic values of the above step (c). The correlation coefficients thereof (r) can be expressed as shown in the list (Tables 3 to 14) which are prepared in the Examples mentioned later, and can be subjected to a test of significance by t-test.

By the above-mentioned method, there is prepared a gene function database equipped with information how the function-known genes are functionally related to each other. The database is inputted into a computer and used for a method of analyzing gene functions which will be mentioned later.

Now the method of analyzing gene functions provided by this application will be illustrated.

The method of analyzing gene functions of this invention using DSPA is a method where function-unkown gene (gₓ) is analyzed using the above gene function database and comprises the following steps (i) to (iii).

Step (i):

There is measured the IC₄₀ value for each drug from the viabilities at various concentrations of a plural number of drugs (D₁, D₂, D₃, . . . Dₙ) for transformed eukaryotic cells overexpressing the unknown gene (gₓ).

Types of the vector for recombination of cDNA of unknown gene (gₓ), the eukaryotic cells and the drugs and measurement of the viabilities are the same as those in the above step (a) for the construction of the gene function database.

Step (ii):

There are calculated the correlation coefficients between the unknown gene (gₓ) and the known genes (g₁, g₂, g₃, . . . gₙ) from the IC₄₀ value of the above step (i) by the same method (steps (b) to (d)) as in the calculation of the correlation coefficients among the known genes (g₁, g₂, g₃, . . . gₙ) of the gene function database.

Step (iii):

There is determined that the function of the known gene showing a significant correlation coefficient to the unknown gene (gₓ) is related to the function of the unknown gene (gₓ).

Thus, in the t-test for the calculation of the correlation coefficient “r”, t={r²(n−2)/(1−r²)}^{1/2} (n: data numbers). Therefore, in case the data for 40 or more kinds of drugs are available, the correlation coefficient “r” is significant when it is not less than 0.4 or not more than −0.4. More preferably, when the case where correlation coefficient “r” is not less than 0.5 or not more than −0.5 is used as a standard, it is possible to clearly specify the relationship among the genes. In the case of each of genes shown in Tables 3 to 14, the gene Ha-ras for example shows a correlation of not less than 0.5 to Ki-ras (r=0.71), N-ras (0.56) and erbB2 (0.53), and shows a correlation of not more than −0.5 to Cip-1 (−0.55), RhoA (−0.55) and C/EBPb (−0.50) whereupon it is noted that Ha-ras is functionally related to those genes. When there is such a high correlation between unknown gene and known gene, it is can be judged that the function of the known gene is related to the function of the unknown gene as well.

Incidentally, the above steps (ii) and (iii) can be quickly processed by a computer. Further, when the data of the function-unknown gene being newly functionally analyzed by the method of analyzing the gene functions are appropriately added to the above-mentioned database, it is now possible to be developed to a database having higher accuracy.

As a result of the method of analyzing gene functions as mentioned above, function of the unknown gene can be quickly decided, and action mechanism of the genetic product can be estimated with a high accuracy. Thus, in most cases, overexpression of gene affects the molecule which is related to the product thereof. The molecule affected as such further affects the surrounding molecules and, as a result, a pathway such as a signal transduction is activated or inactivated. Such a cell shows a different sensitivity to the drugs which act the molecule relating to the pathway. Alternatively, it also shows a different sensitivity to a physiologically active substance acting on the same pathway such as cytokines. On the other hand, it has been confirmed that, in the case of genes where their functional relations have been known already (such as p53 and p21), similar sensitivity to the drugs is noted. It is presumed that there are several decades of main signal transduction pathways and that, even when minor routes accompanied therewith are included, there are about 100 kinds. Accordingly, by constructing a database where result of influence (or result of non-influence) to sensitivity to drugs by overexpression of fuction-known genes (preferably, 100 kinds or more) made into a pattern using the correlation value for each other followed by comparing the result of the overexpression of function-unknown genes with the database, it is possible to identify the pathway concerning the product of the function-unknown genes. Further, when the pathway is investigated, a direct action mechanism of the genetic product can be elucidated with a high accuracy.

The invention of this application will now be illustrated in more detail and specifically by way of the following Examples, although the invention of this application is not limited to the following Examples.

### EXAMPLES

1. Materials

With regard to the function-known genes, the genes shown in the left column of Table 2 were used. With regard to the drugs, those shown in Table 1 were dissolved in DMSO to an extent of 100-fold of the maximum concentration for the search of the drugs and used. With regard to the cells, incubated NIH3T3 or ras-NIH3T3 cells were used.

TABLE 1

| | | |
|---|---|---|
| Tyrosine kinase | | |
| inhibitors | | |
| HMA | herbimycin A | Src, Abl inhibitor |
| erbstatin | | EGFR inhibitor |
| genistein | | Tyr kinase, topo II inhibitor |
| Ser/Thr kinase | | |
| inhibitors | | |
| Staurosporine | | PKC, cdk, MLCK inhibitor |
| K252a | | CaM-kinase inhibitor |
| H-7 | | PKA, PKC, PKG inhibitor |
| GF109203X | | PKC inhibitor |
| Y-27632 | | ROCK inhibitor |
| olomoucine | | cdk inhibitor |
| Phosphatase | | |
| inhibitors | | |
| OA | okadaic acid | PP1, PP2A inhibitor |
| cantharidin | | PP2A inhibitor |
| Na vanadate | sodium vanadate | tyrosine phosphatase inhibitor |
| NaAsO₂ | sodium arsenite | tyrosine phosphatase inhibitor |
| Anti-cancer | | |
| drugs | | |
| MMC | mitomycin C | DNA cross-linker |
| 5-FU | 5-fluorouracil | thymidine synthetase inhibitor |
| CDDP | cisplatin | DNA cross-linker |
| MTX | methotrexate | DHFR inhibitor |
| MIT | mitoxantrone | DNA strand break |
| ACR | aclarubicin | DNA intercalator |
| IFM | ifosfamide | DNA alkylation |
| ACD | actinomycin D | RNA polymerase inhibitor |
| HU | hydroxyurea | ribonucleotide reductase inhibitor |
| 6-TG | 6-thioguanine | purine metabolism inhibitor |
| CPT | camptothecin | topoisomerase I inhibitor |
| PPL | pepleomycin | DNA strand break |
| VCR | vincristine | microtubule depolymerization |
| Protease | | |
| inhibitors | | |
| ALLN | Ac-Leu-Leu- | calpain/proteasome inhibitor |
| | leucinal | |
| ONO-3403 | | trypsin inhibitor |
| ONO-5046 | | elastase inhibitor |
| ICEin-III | ICE inhibitor-III | caspase inhibitor |
| Others | | |
| wortmannin | | P13-K inhibitor |
| manumycin | | farnesyltransferase inhibitor |
| cytochalasin D | | actin polymerization inhibitor |
| ouabain | | Na⁺, K⁺-ATPase inhibitor |
| A23187 | | Ca²⁺ ionophore |
| BAPTA-AM | | Ca²⁺ chelator |
| thapsigargin | | Ca²⁺-APTase inhibitor |
| U73122 | | phospholipase C inhibitor |
| SnPP | | heme oxygenase inhibitor |
| curcumin | | 5-lipoxygenase & cyclooxygenase |
| | | inhibitor |
| forskolin | | adenylate cyclase activator |
| IBMX | | phosphodiesterase inhibitor |
| CHX | cycloheximide | protein synthesis inhibitor |
| SNAP | | NO donor |
| resveratrol | | antioxidant; estrogen-R agonist, |
| | | COX1 inhibitor |
| | | ribonucleotide reductase inhibitor |
| sulindac | | COX inhibitor |
| IMT | indomethacin | COX1/2 inhibitor, phospholipase |
| | | A2 inhibitor |
| NaN₃ | sodium azide | cytochrome a/a3 binding |
| β-elemene | | herb medicine |

2. Methods

(1) Preparation of Cells Overexpressing Genes

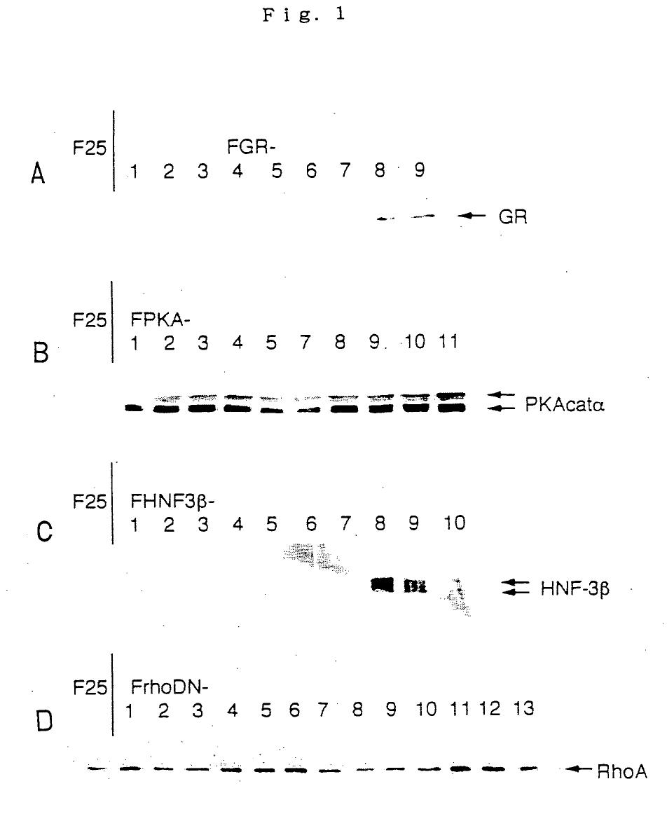

Full-length cDNA of each function-known genes was incorporated into an expression vector (pRc-CMV, etc.) and transfected into NIH3T3 cells by a common method. Transformed cells were isolated using the resistance to G-418 as an index and the expression of the transfected gene in each cell was investigated by Western blotting method to select a highly expressing line. FIG. 1 shows examples of gene expression by Western blotting method.

(2) Measurement of Cell Viabilities by MTT Method

Cell viabilities were measured according to the following procedures.

1) From the 2nd to the 11th rows of a 96-well plate were filled with DMEM (50 μl). The 12th row was filled with 100 μl of DMEM.

2) The 1st row was filled with 98 μl of DMEM.

3) Drugs (2 μl each) were added into the 1st row.

4) Continuous double-dilution was conducted from the 1st to the 9th rows. Fifty μl were taken out from the 9th row and discarded. The plate was preserved in a CO₂ incubator.

5) Cells (each high-expressing cells and the parental cell line) were detached from the medium using a trypsin solution, suspended in 10% CS-DMEM and transferred to a test tube and cell numbers (cell/ml) were counted.

6) The cells were diluted to a concentration of 1×10⁵ cells/ml, and 50 μl of the cell suspension were added to the 1st to the 11th rows of the plate followed by gently stirring.

7) The plate was transferred to the CO₂ incubator and incubated for 3 to 4 days.

8) MTT (5 mg/ml in PBS) (10 μl) was added to the 1st to the 12th rows of the plate and incubated in the CO₂ incubator at 37° C. for 4 hours.

9) A reaction stop solution (0.04N HCl in isopropanol) (150 μl) was added followed by mixing well and being allowed to stand at room temperature for about 2 hours.

10) Coloration of formazan which was a metabolite of the MTT dye was measured using a microplate reader. The measurement wavelength was 574 nm and the reference wavelength was 655 nm. The 10th and the 11th rows of the plate were 100% controls and the 12th row was a 0% control.

(3) Calculation of Logarithmic Values of Ratios of IC₄₀ Values

As an index for the sensitivity of highly gene-expressing cells and the parental cell line to various drugs, each of the IC₄₀ values thereof was specified and ratios of the IC₄₀ values were calculated. FIG. 2 and FIG. 3 are the examples where ratios of IC₄₀ values were calculated from a curve showing the dependency on the concentration of the chemical versus the viability. After that, logarithmic values of ratios of IC₄₀ values were calculated from each gene. Table 2 is a part of the list of the logarithmic values.

TABLE 2

| | GF109203X | Staurosporine | K252a | H-7 | HMA | Genistein | Erbstatin | Ouabain | 6-TG | OA | SnPP | Vanadate | ALLN |
|---|---|---|---|---|---|---|---|---|---|---|---|---|---|
| Ha-ras | −0.39 | 0.30 | 0.65 | 0.23 | −0.35 | −0.35 | 0.08 | 0.32 | −0.19 | 0.11 | 0.28 | −0.33 | 0.15 |
| Ki-ras | −0.40 | 0.38 | 0.15 | 0.28 | −0.14 | 0.00 | 0.04 | 0.18 | −0.30 | 0.04 | 0.18 | −0.30 | 0.11 |
| N-ras | | 0.24 | −0.16 | 0.30 | 0.43 | 0.42 | −0.22 | 0.56 | −0.22 | 0.00 | 0.14 | −0.09 | 0.17 |
| v-src | −0.29 | −0.05 | −0.22 | −0.07 | −0.35 | −0.60 | −0.05 | 0.00 | 0.00 | 0.18 | −0.17 | 0.00 | 0.10 |
| erbB2 | −0.40 | 0.04 | 0.15 | 0.00 | −0.55 | −0.60 | −0.05 | 0.18 | −0.12 | −0.19 | −0.25 | −0.40 | −0.05 |
| p53(wt) | 0.62 | 0.07 | 0.05 | 0.00 | −0.22 | 0.26 | 0.17 | 0.09 | 0.32 | −0.05 | −0.18 | 0.00 | 0.18 |
| Cip-1 | 0.30 | 0.00 | | 0.00 | −0.30 | | | 0.16 | 0.47 | −0.15 | −0.66 | 0.00 | 0.18 |
| p16/INK4A | 0.48 | −0.22 | −0.22 | 0.08 | 0.41 | 0.40 | 0.51 | 0.63 | 0.46 | 0.06 | −0.05 | 0.11 | 0.09 |
| Bax | 0.22 | −0.22 | −0.11 | 0.08 | −0.05 | 0.20 | 0.27 | 0.40 | 0.36 | 0.00 | 0.17 | 0.00 | −0.10 |
| Bax | 0.27 | 0.00 | 0.60 | 0.00 | 0.15 | 0.38 | 0.04 | 0.00 | −0.05 | 0.00 | 0.10 | 0.32 | 0.11 |
| TK | 0.00 | 0.46 | 0.23 | −0.55 | 0.00 | 0.08 | 0.05 | 0.00 | −0.17 | 0.00 | 0.00 | 0.16 | 0.00 |
| m-calpain | 0.35 | 0.00 | 0.13 | 0.15 | 0.00 | 0.18 | 0.00 | 0.05 | 0.29 | −0.08 | −0.07 | −0.10 | 0.00 |
| cAMP-PK-CS | 0.27 | −0.12 | −0.04 | 0.15 | 0.12 | 0.00 | 0.05 | 0.00 | 0.14 | 0.08 | 0.02 | 0.00 | 0.20 |
| calpastatin | | 0.04 | 0.1 | −0.14 | −0.14 | 0.127 | −0.2 | −0.3 | −0.25 | −0.04 | 0.486 | 0.00 | 0.10 |
| CP-antisense | 0.00 | −0.15 | 0.00 | 0.00 | −0.10 | 0.00 | 0.06 | 0.28 | 0.14 | 0.13 | 0.10 | 0.04 | 0.00 |
| DAN | 0.65 | 0.48 | −0.46 | 0.18 | −0.05 | 0.20 | 0.20 | 0.74 | 0.64 | 0.04 | −0.49 | 0.30 | 0.00 |
| Regucalcin | −0.05 | −0.09 | −0.34 | −0.02 | −0.20 | 0.00 | 0.00 | −0.12 | 0.20 | −0.10 | 0.32 | 0.00 | 0.00 |
| cystatin alpha | 0.60 | 0.30 | −0.10 | 0.60 | −0.12 | 0.28 | 0.08 | 1.28 | 0.60 | 0.08 | 0.00 | 0.08 | 0.07 |
| cystatin E | 0.27 | 0.40 | −0.19 | −0.05 | 0.00 | 0.21 | −0.06 | 0.49 | 0.00 | −0.52 | 0.11 | 0.20 | 0.19 |
| caspase-1 | 0.23 | 0.22 | 0.04 | 0.00 | 0.00 | −0.12 | −0.22 | 1.26 | 0.34 | 0.00 | −0.15 | −0.11 | −0.04 |
| caspase-3 | 0.82 | 0.00 | 0.15 | −0.15 | 0.08 | 0.04 | −0.10 | 0.65 | 1.16 | 0.00 | −0.40 | 0.14 | −0.09 |
| caspase-2 | 0.92 | 0.18 | 0.37 | 0.28 | 0.10 | 0.30 | −0.07 | −0.05 | 0.18 | 0.06 | −0.33 | 0.40 | −0.04 |
| RhoA | 0.24 | 0.15 | 0.10 | 0.35 | 0.13 | 0.31 | 0.14 | 0.15 | 0.69 | 0.00 | 0.14 | 0.28 | 0.00 |
| RhoA-DN | | 0.14 | 0.28 | 0.22 | −0.10 | 0.00 | −0.02 | 0.45 | 0.41 | 0.00 | 0.08 | −0.12 | 0.00 |
| PDGF-R | 0.00 | 0.24 | 0.10 | −0.07 | 0.19 | 0.32 | | 0.34 | 0.22 | 0.16 | −0.07 | 0.22 | 0.22 |
| PDGF-R-Del | 0.91 | 0.28 | 0.10 | 0.28 | 0.00 | 0.33 | 0.00 | 0.19 | 0.51 | 0.21 | 0.08 | 0.00 | 0.10 |
| Gluco- | 0.27 | −0.28 | 0.13 | 0.08 | −0.11 | 0.02 | 0.06 | 0.04 | 0.09 | 0.06 | 0.00 | 0.03 | 0.12 |
| corticoid-R | | | | | | | | | | | | | |
| STAT1 | 0.41 | 0.00 | 0.15 | 0.08 | −0.05 | 0.10 | 0.04 | 0.04 | 0.20 | 0.14 | 0.08 | 0.00 | 0.17 |
| CBP8 | 0.16 | 0.19 | 0.28 | −0.06 | 0.09 | 0.10 | 0.14 | 0.58 | 0.38 | 0.12 | 0.56 | 0.03 | 0.11 |
| PKCalpha-KN | 0.22 | 0.00 | 0.16 | 0.14 | 0.09 | 0.16 | | 0.00 | 0.26 | 0.09 | −0.14 | 0.23 | 0.11 |
| PKCalpha-KN | 0.62 | −0.10 | 0.02 | 0.20 | −0.10 | 0.24 | 0.00 | 0.06 | 0.38 | 0.00 | 0.00 | 0.08 | −0.01 |
| PKCepsilon- | −0.17 | −0.07 | 0.00 | −0.21 | −0.11 | 0.00 | −0.12 | −0.15 | 0.00 | 0.00 | 0.27 | 0.09 | 0.07 |
| KN | | | | | | | | | | | | | |
| PKCepsilon- | 0.32 | −0.10 | −0.09 | 0.16 | 0.09 | 0.19 | 0.00 | −0.12 | 0.00 | 0.00 | 0.24 | 0.18 | 0.19 |
| KN | | | | | | | | | | | | | |
| TPA (PKC | 0.09 | −0.12 | −0.13 | 0.00 | −0.07 | −0.09 | 0.00 | −0.08 | 0.00 | 0.12 | −0.20 | −0.03 | −0.09 |
| downreg | | | | | | | | | | | | | |
| ERK-DN | 0.48 | 0.06 | 0.05 | 0.14 | 0.00 | 0.00 | 0.13 | 0.48 | 0.32 | 0.00 | 0.00 | 0.00 | 0.00 |
| JNK-DN | 0.58 | 0.14 | −0.28 | 0.09 | −0.14 | −0.10 | 0.11 | −0.08 | 0.00 | −0.07 | −0.11 | −0.17 | −0.24 |
| p38-DN | 0.58 | −0.05 | −0.15 | 0.10 | −0.05 | 0.00 | 0.06 | 0.41 | 0.33 | 0.12 | 0.10 | −0.04 | 0.04 |
| HSP4O | −0.07 | 0.30 | 0.07 | 0.15 | 0.00 | 0.00 | 0.00 | 0.00 | 0.50 | −0.02 | 0.28 | −0.03 | 0.18 |
| HSP9O | | 0.22 | 0.56 | −0.03 | 0.40 | 0.41 | 0.26 | 0.92 | 0.57 | 0.07 | | 0.02 | 0.16 |
| CaMKIIa | 0.04 | 0.12 | 0.04 | 0.00 | 0.00 | −0.15 | 0.14 | 0.23 | 0.00 | 0.00 | 0.24 | −0.13 | 0.00 |
| CaMKIIa- | | −0.04 | 0.10 | 0.13 | 0.51 | 0.23 | 0.21 | 0.60 | 0.62 | 0.14 | 0.30 | 0.01 | 0.20 |
| Active | | | | | | | | | | | | | |
| HNF1 | 0.345 | 0 | −0.207 | 0.149 | 0.161 | 0.234 | 0.391 | 0.103 | 0.46 | 0 | 0.138 | 0.046 | 0.16 |
| HNF3b | 0.21 | 0.00 | −0.21 | −0.11 | −0.18 | −0.21 | −0.03 | −0.23 | 0.03 | 0.08 | 0.24 | 0.22 | 0.28 |
| HNF4 | 0.40 | 0.04 | −0.22 | 0.09 | −0.08 | −0.15 | 2.00 | −0.08 | 0.00 | 0.08 | 0.46 | −0.03 | −0.15 |
| coup | 0.46 | 0.08 | 0.00 | 0.28 | 0.42 | 0.44 | 0.33 | 0.49 | 0.45 | 0.00 | 0.51 | −0.10 | 0.06 |
| C/EBPa | 0.00 | 0.00 | −0.48 | −0.35 | 0.00 | −0.43 | 0.11 | 0.00 | 0.00 | 0.12 | 0.00 | −0.02 | 0.00 |
| C/EBPb | | −0.23 | 0.25 | 0.00 | 0.45 | 0.19 | 0.48 | 0.00 | 0.30 | 0.21 | −0.50 | | 0.31 |
| p33/ING1 | −0.28 | 0.62 | 0.28 | 0.00 | 0.38 | 0.78 | 0.11 | 0.10 | 0.46 | 0.11 | −0.23 | −0.30 | 0.08 |
| T.Tn | −0.10 | 0.00 | 0.00 | 0.00 | 0.00 | −0.25 | 0.48 | 0.28 | 0.00 | −0.16 | 0.00 | 0.46 | 0.48 |

| | | | ICEin- | | | Thapsi- | BAPTA- | | | | | | b- |
|---|---|---|---|---|---|---|---|---|---|---|---|---|---|
| | 3403 | 5046 | 3 | Forskolin | U73122 | gargin | AM | Wortmannin | Manumycin | A23187 | NaAsO2 | CHX | elemene |
| Ha-ras | −0.22 | 0.00 | 0.32 | 0.26 | −0.19 | 0.00 | −0.20 | 0.34 | −0.30 | −0.28 | 0.04 | −0.19 | −0.26 |
| Ki-ras | −0.30 | 0.51 | 0.30 | 0.36 | −0.60 | −0.05 | −0.15 | 0.34 | −0.24 | 0.06 | 0.08 | −0.43 | −0.10 |
| N-ras | −0.17 | 0.23 | 0.38 | 0.54 | −0.54 | 0.08 | 0.00 | 0.14 | −0.09 | 0.20 | −0.19 | −0.42 | 0.07 |
| v-src | −0.05 | 0.51 | −0.14 | −0.46 | −0.59 | 0.32 | −0.22 | 0.08 | 0.00 | −0.19 | 0.11 | −0.32 | −0.35 |
| erbB2 | −0.15 | 0.80 | 0.18 | 0.28 | −0.96 | 0.11 | −0.40 | 0.16 | −0.05 | −0.07 | −0.15 | −0.64 | −0.46 |
| p53(wt) | 0.04 | | 0.23 | 0.98 | 0.11 | −0.05 | 0.00 | 0.40 | 0.12 | 0.30 | 0.20 | 0.85 | 0.00 |
| Cip-1 | 0.04 | −0.14 | 0.08 | | 0.08 | 0.04 | 0.00 | | 0.12 | 0.04 | 0.30 | 1.77 | 0.00 |
| p16/INK4A | 0.29 | 0.22 | 0.15 | 0.70 | 0.28 | −0.64 | 0.20 | 0.30 | 0.08 | 0.00 | 0.16 | 0.04 | 0.12 |
| Bax | 0.12 | −0.07 | −0.12 | 0.00 | 0.00 | −0.21 | 0.15 | 0.14 | −0.07 | 0.00 | −0.10 | −0.08 | 0.14 |
| Bax | 0.00 | 0.00 | 0.00 | −0.26 | 0.00 | 0.20 | 0.00 | 0.10 | 0.06 | −0.09 | 0.34 | 0.00 | 0.00 |
| TK | 0.09 | −0.24 | −0.15 | 0.56 | 0.00 | 0.06 | 0.00 | 0.00 | 0.00 | 0.00 | 0.18 | −0.13 | −0.14 |
| m-calpain | 0.00 | −0.04 | −0.06 | 0.25 | −0.14 | 0.05 | 0.00 | 0.18 | 0.05 | 0.26 | −0.16 | 0.22 | 0.00 |
| cAMP-PK- | 0.00 | 0.00 | 0.00 | 0.51 | −0.18 | 0.05 | −0.10 | −0.07 | 0.08 | −0.08 | 0.07 | 0.23 | −0.10 |
| CS | | | | | | | | | | | | | |
| calpastatin | 0.00 | −0.1 | −0.088 | −0.09 | −0.313 | 0.046 | 0 | −0.14 | 0.02 | −0.12 | 0 | 0.31 | 0.18 |
| CP-antisense | 0.00 | −0.06 | −0.19 | −0.05 | −0.10 | −0.30 | 0.06 | −0.12 | 0.00 | −0.36 | 0.02 | 0.00 | 0.00 |
| DAN | 0.08 | 0.26 | 0.00 | 0.88 | 0.36 | 0.18 | 0.70 | 0.52 | 0.00 | 0.40 | −0.15 | 0.72 | 0.04 |
| Regucalcin | 0.12 | −0.38 | 0.00 | −0.10 | −0.66 | 0.20 | −0.39 | −0.28 | 0.04 | 0.03 | 0.03 | 0.08 | −0.09 |
| cystatin | −0.10 | | 0.32 | 0.72 | 0.04 | 0.04 | 0.34 | 0.45 | −0.19 | 0.26 | −0.05 | 0.34 | 0.08 |
| alpha | | | | | | | | | | | | | |
| cystatin E | −0.15 | 0.36 | 0.07 | 0.29 | −0.07 | 0.00 | −0.02 | 0.00 | 0.00 | 0.22 | 0.15 | 0.00 | 0.07 |
| caspase-1 | −0.06 | 0.40 | 0.11 | 0.34 | 0.53 | −0.30 | 0.60 | 0.20 | −0.06 | −0.22 | −0.21 | −0.05 | 0.11 |
| caspase-3 | 0.00 | 0.40 | 0.13 | 1.12 | 0.53 | −0.17 | 0.60 | 0.45 | 0.04 | 0.28 | 0.11 | 0.10 | 0.28 |
| caspase-2 | 0.15 | 0.51 | 0.04 | −0.34 | −0.02 | −0.07 | 0.23 | 0.52 | 0.08 | 0.48 | 0.18 | 0.74 | 0.26 |
| RhoA | 0.00 | 0.11 | 0.19 | 0.47 | 0.12 | 0.26 | 0.00 | 0.24 | −0.04 | 0.26 | 0.07 | 0.14 | 0.00 |
| RhoA-DN | 0.22 | 0.00 | 0.00 | −0.36 | −0.35 | 0.06 | 0.09 | 0.00 | −0.13 | 0.03 | 0.00 | 0.22 | |
| PDGF-R | 0.00 | 0.36 | 0.20 | 0.16 | −0.11 | 0.29 | 0.29 | 0.00 | 0.17 | 0.31 | 0.14 | 0.51 | 0.00 |
| PDGF-R-Del | 0.22 | −0.07 | 0.00 | 0.51 | 0.00 | 0.24 | 0.31 | 0.07 | 0.09 | 0.15 | 0.00 | 0.39 | 0.16 |
| Gluco | 0.10 | 0.00 | 0.00 | 0.33 | 0.05 | 0.20 | 0.10 | 0.09 | 0.09 | 0.28 | 0.16 | 0.09 | 0.00 |
| corticoid-R | | | | | | | | | | | | | |
| STAT1 | 0.16 | 0.05 | 0.09 | 0.51 | 0.18 | 0.30 | 0.10 | 0.00 | 0.00 | 0.31 | 0.16 | 0.00 | 0.05 |
| CBP8 | 0.08 | 0.12 | 0.18 | 0.40 | 0.18 | 0.12 | 0.00 | 0.14 | 0.09 | 0.22 | 0.21 | 0.00 | 0.06 |
| PKCalpha- | 0.04 | 0.10 | 0.12 | −0.47 | −0.33 | 0.10 | 0.43 | 0.41 | 0.00 | 0.22 | 0.16 | 0.30 | 0.07 |
| KN | | | | | | | | | | | | | |
| PKCalpha- | 0.11 | −0.05 | 0.33 | 0.00 | −0.97 | 0.12 | −0.42 | −0.06 | 0.16 | −0.05 | 0.22 | 0.29 | −0.07 |
| KN | | | | | | | | | | | | | |
| PKCepsilon- | 0.04 | −0.21 | −0.21 | 0.00 | −0.21 | 0.09 | 0.00 | −0.15 | −0.11 | −0.28 | −0.07 | 0.06 | 0.00 |
| KN | | | | | | | | | | | | | |
| PKCepsilon- | 0.00 | 0.00 | 0.33 | 0.00 | −0.38 | 0.20 | 0.00 | −0.28 | 0.16 | 0.15 | 0.35 | 0.20 | −0.23 |
| KN | | | | | | | | | | | | | |
| TPA | 0.10 | −0.10 | −0.09 | −0.17 | −0.12 | −0.23 | −0.03 | 0.00 | 0.03 | −0.22 | −0.20 | 0.00 | 0.00 |
| (PKCdownr | | | | | | | | | | | | | |
| ERK-DN | 0.10 | 0.00 | 0.00 | 0.08 | 0.00 | −0.15 | 0.12 | −0.11 | −0.05 | 0.00 | 0.16 | 0.08 | 0.33 |
| JNK-DN | 0.22 | −0.09 | 0.00 | −0.22 | −0.07 | 0.24 | 0.06 | −0.09 | −0.05 | −0.08 | −0.07 | 0.00 | −0.04 |
| p38-DN | 0.14 | −0.04 | −0.11 | −0.46 | 0.00 | 0.02 | 0.00 | −0.18 | 0.00 | −0.12 | 0.00 | 0.04 | 0.05 |
| HSP4O | 0.17 | −0.15 | 0.11 | −0.39 | −0.20 | 0.18 | 0.05 | −0.07 | −0.06 | −0.16 | −0.02 | 0.00 | 0.02 |
| HSP9O | 0.36 | 0.52 | 0.22 | 0.94 | 0.18 | 0.09 | 0.60 | 0.18 | −0.13 | 0.28 | 0.10 | 0.07 | 0.05 |
| CaMKIIa | 0.00 | 0.00 | 0.00 | 0.00 | −0.05 | 0.09 | −0.12 | −0.15 | 0.00 | 0.00 | 0.00 | 0.04 | 0.16 |
| CaMKIIa- | 0.22 | 0.35 | 0.22 | 0.43 | 0.00 | 0.03 | 0.18 | 0.18 | 0.03 | 0.18 | 0.02 | −0.09 | |
| Active | | | | | | | | | | | | | |
| HNF1 | 0.14 | 0.05 | 0.20 | 1.15 | 0.07 | 0.16 | 0.20 | 0.16 | 0.06 | 0.35 | −0.14 | 0.38 | 0.02 |
| HNF3b | 0.22 | −0.25 | −0.09 | 0.00 | −0.31 | 0.24 | −0.07 | 0.24 | −0.06 | −0.16 | 0.00 | 0.07 | −0.07 |
| HNF4 | −0.09 | 0.00 | 0.00 | 0.00 | 0.00 | 0.00 | −0.10 | 0.14 | 0.00 | 0.19 | 0.00 | 0.22 | 0.33 |
| coup | 0.11 | −0.04 | −0.11 | 0.78 | 0.00 | −0.30 | 0.09 | 0.10 | 0.09 | −0.12 | −0.19 | 0.12 | 0.52 |
| C/EBPa | −0.41 | 0.00 | −0.46 | 0.00 | 0.00 | −0.09 | −0.62 | 0.00 | 0.00 | 0.00 | −0.40 | 0.00 | 0.00 |
| C/EBPb | 0.36 | 0.17 | 0.35 | 0.93 | 0.10 | 0.29 | 0.81 | 0.34 | 0.47 | 0.25 | 0.41 | 0.37 | 0.50 |
| p33/ING1 | 0.00 | −0.25 | −0.15 | 0.63 | 0.28 | 0.49 | 0.00 | 0.00 | 0.18 | 0.26 | −0.07 | −0.30 | −0.14 |
| T.Tn | 0.00 | 0.00 | 0.30 | 0.53 | −0.26 | 0.32 | 0.11 | 0.36 | 0.28 | −0.22 | −0.05 | 0.00 | −0.14 |

(4) Calculation of Correlation Coefficients by Logarithmic Values

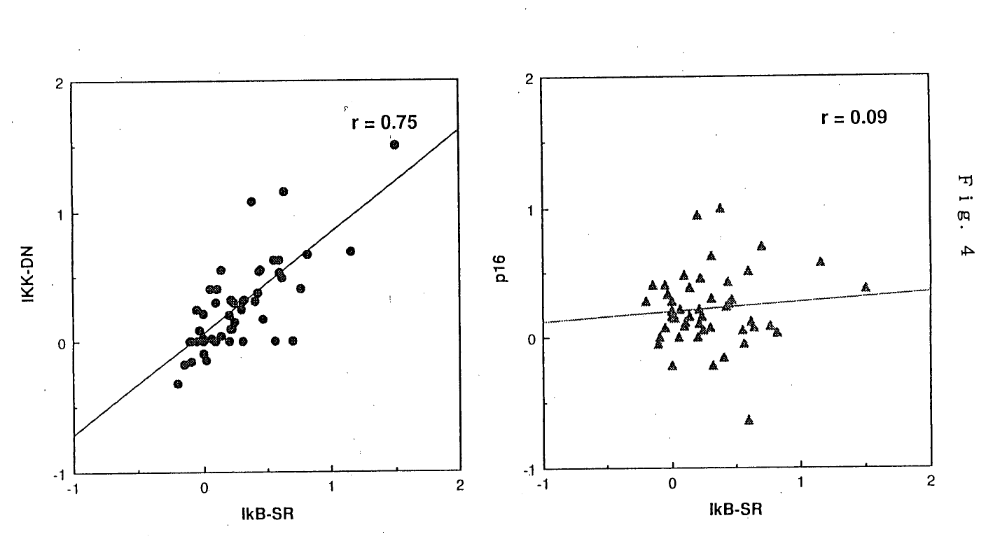

In the list of the logarithmic values shown in Table 2, calculation of correlation coefficients by t-test was carried out in each line. FIG. 4 shows graphs where sensitivity ratios of IkB-SR and IKK-DN— and p16-introduced cells to chemicals were logarithmically plotted, and, between IkB and IKK which were functionally correlated, there was a high correlation (r=0.75) while, between IkB and p16 which were not related, the correlation coefficient was 0.09 with no significance. In Tables 3 to 14, the list of all correlation coefficients is shown by dividing into 12.

TABLE 3

| | Ha-ras | Ki-ras | N-ras | v-src | erbB2 | abl | Ras-N-17 | Rb | p53(wt) | Cip-1 | p16/INK4A | HSP4O | HSP9O | Hsdj | DnaJ-61 | Bax (N) | Bax (F) | IkB-TDN | IKK-DN |
|---|---|---|---|---|---|---|---|---|---|---|---|---|---|---|---|---|---|---|---|
| Ha-ras | 1.00 | | | | | | | | | | | | | | | | | | |
| Ki-ras | 0.71 | 1.00 | | | | | | | | | | | | | | | | | |
| N-ras | 0.56 | 0.56 | 1.00 | | | | | | | | | | | | | | | | |
| v-src | 0.36 | 0.61 | 0.08 | 1.00 | | | | | | | | | | | | | | | |
| erbB2 | 0.53 | 0.76 | 0.35 | 0.64 | 1.00 | | | | | | | | | | | | | | |
| abl | −0.16 | −0.09 | −0.05 | −0.17 | −0.07 | 1.00 | | | | | | | | | | | | | |
| Ras-N-17 | 0.11 | −0.06 | 0.12 | −0.24 | −0.27 | 0.51 | 1.00 | | | | | | | | | | | | |
| Rb | 0.02 | 0.06 | −0.15 | −0.15 | −0.02 | −0.16 | −0.01 | 1.00 | | | | | | | | | | | |
| p53(wt) | −0.29 | −0.30 | −0.34 | −0.44 | −0.17 | 0.25 | 0.30 | 0.18 | 1.00 | | | | | | | | | | |
| Cip-1 | −0.55 | −0.37 | −0.37 | −0.30 | −0.31 | 0.19 | 0.18 | −0.12 | 0.57 | 1.00 | | | | | | | | | |
| p16/INK4A | −0.36 | −0.21 | −0.18 | −0.26 | −0.17 | 0.18 | 0.06 | −0.04 | 0.41 | 0.22 | 1.00 | | | | | | | | |
| HSP4O | −0.14 | −0.13 | −0.33 | 0.01 | −0.01 | 0.03 | −0.16 | 0.45 | 0.00 | 0.17 | −0.20 | 1.00 | | | | | | | |
| HSP9O | −0.23 | −0.10 | −0.32 | −0.17 | 0.00 | 0.32 | −0.01 | 0.43 | 0.42 | 0.15 | 0.46 | 0.21 | 1.00 | | | | | | |
| Hsdj | −0.33 | −0.05 | −0.18 | −0.11 | −0.01 | 0.39 | 0.19 | 0.19 | 0.16 | 0.34 | −0.04 | 0.34 | 0.33 | 1.00 | | | | | |
| DnaJ-61 | −0.15 | −0.05 | 0.05 | −0.24 | 0.03 | 0.19 | 0.10 | 0.21 | 0.30 | −0.05 | 0.17 | −0.05 | 0.26 | 0.40 | 1.00 | | | | |
| Bax(N) | −0.36 | −0.35 | −0.01 | −0.34 | −0.27 | 0.09 | −0.22 | −0.10 | 0.08 | 0.25 | 0.10 | 0.21 | 0.09 | 0.15 | −0.06 | 1.00 | | | |
| Bax(F) | −0.20 | 0.00 | −0.11 | 0.02 | −0.06 | −0.07 | −0.10 | 0.11 | 0.08 | 0.18 | 0.56 | 0.17 | 0.22 | 0.01 | −0.17 | 0.19 | 1.00 | | |
| 1kB-SR | −0.24 | −0.28 | −0.12 | −0.39 | −0.28 | −0.05 | 0.16 | 0.18 | 0.52 | 0.44 | 0.09 | 0.04 | 0.23 | 0.29 | 0.13 | 0.12 | 0.09 | 1.00 | |
| IKK-DN | −0.58 | −0.49 | −0.35 | −0.48 | −0.34 | 0.18 | 0.03 | 0.12 | 0.45 | 0.62 | 0.16 | 0.25 | 0.28 | 0.41 | 0.16 | 0.39 | 0.16 | 0.75 | 1.00 |
| PDGF-R | −0.14 | 0.16 | 0.18 | 0.30 | 0.13 | 0.35 | 0.13 | −0.20 | 0.11 | 0.26 | −0.06 | −0.02 | 0.32 | 0.10 | 0.05 | 0.02 | −0.06 | 0.11 | 0.16 |
| PDGF-R-Del | −0.38 | −0.16 | −0.37 | −0.19 | −0.17 | 0.19 | 0.14 | 0.34 | 0.45 | 0.43 | 0.38 | 0.11 | 0.62 | 0.26 | 0.30 | 0.02 | 0.24 | 0.24 | 0.44 |
| Glucocorticoid-R | 0.07 | −0.08 | 0.06 | −0.19 | −0.04 | −0.02 | −0.05 | −0.06 | 0.17 | 0.04 | 0.10 | −0.25 | 0.32 | 0.00 | 0.26 | 0.14 | −0.03 | 0.14 | 0.06 |
| RhoA | −0.10 | −0.04 | 0.11 | −0.17 | −0.07 | 0.17 | 0.07 | 0.16 | 0.33 | 0.10 | 0.18 | 0.25 | 0.43 | 0.09 | 0.16 | 0.38 | 0.26 | 0.25 | 0.17 |
| RhoA-DN | −0.55 | −0.35 | −0.47 | −0.22 | −0.16 | 0.10 | −0.05 | 0.26 | 0.29 | 0.53 | 0.23 | 0.51 | 0.54 | 0.50 | 0.16 | 0.32 | 0.24 | 0.32 | 0.63 |
| CaMKIIa | 0.34 | 0.05 | 0.15 | −0.10 | 0.06 | −0.32 | −0.03 | 0.29 | −0.16 | −0.23 | −0.16 | 0.08 | −0.01 | −0.10 | 0.13 | −0.20 | −0.04 | 0.23 | 0.10 |
| CaMKIIa-Active | −0.08 | 0.12 | 0.14 | −0.06 | 0.15 | −0.10 | −0.18 | 0.23 | 0.03 | 0.01 | 0.41 | 0.04 | 0.67 | 0.16 | 0.14 | −0.17 | 0.24 | 0.10 | 0.07 |
| PKCalpha-KN | −0.12 | −0.13 | −0.11 | 0.13 | −0.09 | 0.11 | 0.04 | −0.10 | −0.02 | 0.12 | 0.01 | 0.29 | 0.08 | 0.06 | −0.27 | 0.44 | 0.27 | −0.04 | 0.08 |
| PKCalpha-KN | −0.07 | 0.10 | 0.05 | 0.16 | 0.32 | 0.08 | 0.03 | 0.19 | 0.35 | 0.31 | 0.03 | 0.36 | 0.00 | 0.13 | 0.12 | 0.18 | 0.13 | 0.05 | 0.25 |
| PKCepsilon-KN | −0.33 | −0.41 | −0.36 | −0.16 | −0.28 | 0.10 | −0.17 | 0.23 | 0.21 | −0.05 | 0.18 | 0.30 | 0.27 | −0.08 | 0.05 | 0.48 | 0.15 | 0.17 | 0.32 |
| PKCepsilon-KN | −0.09 | 0.12 | 0.19 | 0.22 | 0.16 | −0.06 | −0.16 | −0.06 | 0.06 | 0.11 | −0.1 5 | 0.13 | −0.25 | 0.00 | 0.21 | 0.18 | −0.13 | −0.19 | −0.10 |
| TPA (PKC-) | −0.05 | −0.20 | −0.23 | −0.06 | −0.09 | 0.20 | 0.06 | −0.01 | 0.18 | 0.08 | 0.12 | 0.16 | 0.15 | 0.10 | −0.13 | −0.02 | 0.04 | −0.04 | 0.13 |
| Akt-DN | 0.05 | 0.02 | 0.11 | 0.10 | 0.09 | 0.19 | 0.40 | −0.15 | 0.36 | 0.17 | −0.03 | −0.04 | −0.05 | 0.19 | 0.18 | −0.23 | −0.05 | 0.37 | 0.02 |
| ERK-DN | −0.40 | −0.34 | −0.51 | −0.23 | −0.22 | 0.14 | 0.15 | 0.25 | 0.45 | 0.39 | 0.50 | 0.27 | 0.66 | 0.24 | 0.22 | 0.08 | 0.38 | 0.22 | 0.40 |
| JNK-DN | −0.21 | −0.20 | −0.28 | −0.03 | −0.16 | 0.05 | 0.23 | 0.02 | 0.21 | 0.37 | 0.02 | 0.21 | 0.09 | 0.14 | −0.06 | 0.02 | 0.16 | 0.19 | 0.31 |
| p38-DN | −0.30 | −0.30 | −0.35 | −0.09 | −0.19 | −0.06 | 0.08 | 0.13 | 0.28 | 0.46 | 0.18 | 0.48 | 0.22 | 0.07 | −0.10 | 0.10 | 0.42 | 0.23 | 0.40 |
| cAMP-PK-CS | 0.08 | 0.19 | −0.02 | 0.17 | 0.23 | 0.14 | −0.29 | 0.18 | 0.17 | −0.06 | 0.20 | 0.12 | 0.29 | 0.04 | 0.13 | 0.06 | 0.20 | −0.10 | −0.07 |

TABLE 4

| | Ha-ras | Ki-ras | N-ras | v-src | erbB2 | abl | Ras-N-17 | Rb | p53(wt) | Cip-1 | p16/INK4A | HSP40 | HSP90 | Hsdj | DnaJ-61 | Bax (N) | Bax (F) | IkB-TDN | IKK-DN |
|---|---|---|---|---|---|---|---|---|---|---|---|---|---|---|---|---|---|---|---|
| 14-3-3zWT | −0.16 | −0.23 | −0.30 | −0.06 | −0.02 | −0.12 | −0.03 | 0.34 | 0.21 | −0.11 | −0.02 | 0.28 | 0.14 | 0.19 | 0.13 | −0.01 | 0.02 | 0.23 | 0.26 |
| TK | 0.28 | 0.13 | 0.25 | −0.10 | 0.08 | 0.11 | 0.26 | 0.16 | 0.03 | −0.14 | −0.19 | −0.21 | −0.06 | −0.04 | −0.12 | −0.12 | −0.20 | 0.16 | −0.03 |
| caspase-1 | 0.16 | 0.03 | 0.20 | −0.13 | 0.05 | 0.05 | 0.18 | −0.13 | 0.05 | −0.04 | 0.28 | −0.14 | 0.37 | −0.17 | 0.10 | −0.16 | 0.05 | −0.11 | −0.10 |
| caspase-3 | −0.23 | −0.30 | −0.23 | −0.34 | −0.14 | 0.24 | 0.19 | 0.07 | 0.52 | 0.18 | 0.52 | 0.03 | 0.63 | 0.06 | 0.27 | 0.15 | 0.31 | 0.09 | 0.20 |
| caspase-2 | −0.45 | −0.36 | −0.43 | −0.18 | −0.26 | 0.12 | 0.25 | −0.03 | 0.36 | 0.47 | 0.38 | 0.08 | 0.19 | 0.20 | −0.07 | 0.35 | 0.38 | 0.09 | 0.36 |
| cystatin alpha | 0.12 | 0.17 | 0.45 | −0.07 | 0.27 | 0.13 | 0.24 | 0.04 | 0.39 | 0.15 | 0.42 | −0.02 | 0.51 | 0.04 | 0.32 | −0.03 | 0.49 | 0.12 | 0.10 |
| cystatin E | −0.13 | −0.02 | 0.06 | −0.16 | 0.02 | 0.14 | 0.33 | 0.20 | 0.51 | 0.25 | 0.28 | 0.08 | 0.55 | 0.15 | 0.19 | 0.04 | 0.06 | 0.37 | 0.24 |
| u-calpain | −0.31 | −0.27 | −0.13 | −0.39 | −0.23 | 0.00 | 0.27 | 0.13 | 0.49 | 0.67 | 0.07 | 0.21 | 0.18 | 0.40 | 0.28 | 0.07 | 0.14 | 0.53 | 0.56 |
| m-calpain | −0.30 | −0.18 | −0.05 | −0.35 | −0.10 | 0.22 | −0.05 | 0.16 | 0.41 | 0.27 | 0.14 | 0.25 | 0.30 | 0.26 | 0.25 | 0.38 | 0.19 | 0.23 | 0.41 |
| calpain 30 K (N) | 0.10 | 0.30 | 0.07 | 0.68 | 0.27 | 0.17 | 0.03 | −0.15 | −0.31 | 0.09 | −0.54 | 0.17 | −0.58 | 0.36 | −0.24 | 0.17 | −0.56 | −0.21 | −0.16 |
| calpain 30 K (F) | −0.15 | −0.14 | −0.08 | −0.19 | −0.03 | 0.02 | 0.17 | 0.26 | 0.29 | 0.30 | 0.00 | 0.17 | 0.28 | 0.44 | 0.14 | −0.06 | −0.05 | 0.49 | 0.39 |
| calpastatin | 0.35 | 0.30 | 0.38 | 0.14 | 0.09 | −0.31 | 0.00 | 0.22 | −0.36 | −0.28 | −0.44 | 0.00 | −0.57 | −0.15 | −0.01 | −0.01 | −0.10 | −0.06 | −0.20 |
| CP-antisense | −0.20 | −0.17 | −0.45 | −0.04 | −0.08 | 0.12 | 0.11 | 0.31 | 0.35 | 0.17 | 0.44 | 0.38 | 0.51 | 0.11 | 0.20 | 0.11 | 0.38 | 0.11 | 0.21 |
| BH | −0.17 | −0.27 | −0.06 | −0.37 | −0.33 | −0.29 | 0.03 | 0.11 | 0.19 | 0.29 | 0.01 | 0.02 | 0.02 | 0.11 | −0.04 | 0.14 | 0.06 | 0.72 | 0.57 |
| DAN | −0.45 | −0.45 | −0.37 | −0.33 | −0.24 | 0.27 | 0.24 | 0.13 | 0.44 | 0.35 | 0.46 | 0.21 | 0.43 | 0.28 | 0.20 | 0.24 | 0.48 | 0.15 | 0.38 |
| Regucalcin | −0.22 | −0.12 | −0.02 | −0.15 | −0.05 | −0.10 | −0.05 | 0.26 | 0.32 | 0.39 | 0.02 | 0.36 | −0.08 | 0.20 | 0.20 | 0.13 | 0.08 | 0.39 | 0.37 |
| CathL-mut | −0.03 | −0.14 | −0.16 | −0.09 | −0.24 | −0.36 | −0.17 | 0.51 | 0.00 | −0.20 | −0.09 | 0.19 | −0.09 | −0.23 | −0.22 | 0.20 | 0.06 | 0.16 | 0.11 |
| STAT1 | 0.13 | 0.01 | 0.09 | −0.18 | 0.00 | 0.06 | 0.02 | 0.12 | 0.29 | 0.01 | 0.05 | −0.12 | 0.41 | 0.03 | 0.21 | −0.01 | −0.04 | 0.19 | 0.01 |
| CBP | −0.25 | −0.21 | −0.32 | −0.30 | −0.07 | −0.14 | −0.18 | 0.40 | 0.28 | 0.04 | 0.43 | 0.10 | 0.70 | 0.07 | 0.32 | 0.08 | 0.10 | 0.26 | 0.34 |
| P/CAF | −0.25 | −0.18 | −0.08 | −0.40 | −0.08 | 0.60 | 0.41 | 0.16 | 0.24 | 0.35 | −0.02 | 0.13 | 0.14 | 0.51 | 0.18 | 0.12 | −0.21 | 0.15 | 0.43 |
| HNF1 | −0.05 | −0.12 | −0.01 | −0.34 | −0.05 | 0.15 | 0.20 | 0.26 | 0.60 | 0.07 | 0.34 | −0.14 | 0.30 | −0.01 | 0.43 | −0.34 | 0.07 | 0.36 | 0.14 |
| HNF3b | −0.13 | −0.27 | −0.19 | 0.02 | −0.11 | −0.09 | 0.09 | 0.24 | 0.24 | 0.09 | −0.03 | 0.32 | −0.10 | −0.05 | −0.09 | 0.19 | 0.05 | 0.27 | 0.16 |
| HNF4 | 0.06 | −0.04 | −0.18 | 0.00 | −0.01 | −0.18 | −0.03 | −0.03 | 0.08 | −0.06 | 0.14 | −0.09 | −0.12 | 0.01 | 0.17 | −0.22 | 0.17 | 0.20 | 0.16 |
| coup | −0.25 | −0.12 | −0.17 | −0.40 | −0.18 | 0.06 | 0.13 | 0.57 | 0.52 | 0.19 | 0.54 | 0.17 | 0.62 | 0.21 | 0.30 | −0.06 | 0.43 | 0.42 | 0.38 |
| C/EBPa | −0.13 | −0.04 | −0.09 | −0.07 | 0.16 | 0.26 | 0.21 | 0.19 | 0.26 | 0.30 | 0.23 | 0.16 | 0.36 | 0.36 | 0.21 | 0.02 | 0.22 | 0.02 | 0.20 |
| C/EBPb | −0.50 | −0.25 | −0.50 | −0.04 | −0.08 | 0.27 | 0.00 | 0.08 | 0.45 | 0.37 | 0.25 | 0.03 | 0.53 | 0.33 | 0.16 | −0.05 | 0.06 | 0.16 | 0.28 |
| per-1 | −0.07 | −0.18 | −0.19 | 0.24 | 0.03 | −0.03 | 0.24 | −0.17 | 0.16 | 0.37 | −0.10 | 0.27 | −0.14 | 0.06 | −0.25 | 0.05 | 0.04 | 0.06 | 0.07 |
| p33/ING1 | −0.23 | −0.03 | −0.15 | −0.21 | 0.04 | 0.29 | −0.11 | 0.41 | 0.14 | 0.17 | 0.07 | 0.18 | 0.34 | 0.26 | 0.21 | −0.02 | −0.02 | −0.05 | 0.21 |
| T.Tn | 0.47 | 0.42 | 0.35 | 0.27 | 0.37 | −0.13 | 0.06 | −0.08 | −0.21 | −0.20 | −0.22 | −0.14 | −0.19 | −0.12 | −0.08 | −0.37 | −0.05 | −0.01 | −0.24 |
| CD44/H-CAM | −0.15 | 0.04 | −0.26 | 0.15 | 0.09 | −0.01 | −0.31 | 0.34 | −0.02 | 0.01 | −0.18 | 0.37 | 0.25 | 0.40 | 0.23 | 0.11 | 0.07 | −0.06 | 0.03 |
| CD44/3E | −0.22 | −0.13 | −0.39 | 0.03 | −0.10 | 0.09 | −0.05 | −0.03 | −0.05 | 0.15 | −0.06 | −0.03 | 0.13 | 0.20 | 0.08 | −0.16 | −0.22 | 0.04 | 0.14 |
| CD44/3s | −0.13 | −0.11 | −0.52 | −0.03 | −0.11 | 0.18 | 0.00 | 0.25 | 0.06 | 0.12 | −0.13 | 0.16 | 0.08 | 0.22 | 0.00 | −0.14 | −0.02 | 0.25 | 0.26 |
| Jagged-1 | 0.16 | 0.04 | 0.05 | 0.01 | −0.14 | −0.48 | −0.17 | 0.34 | −0.17 | −0.12 | −0.05 | 0.06 | 0.23 | −0.25 | −0.16 | −0.04 | 0.11 | 0.00 | −0.09 |
| p94-WT | −0.37 | −0.26 | −0.26 | −0.32 | −0.25 | −0.10 | 0.00 | 0.34 | 0.27 | 0.11 | 0.04 | 0.12 | 0.13 | 0.24 | 0.11 | −0.02 | 0.00 | 0.28 | 0.37 |
| p94-mut | −0.23 | −0.07 | 0.02 | 0.03 | −0.14 | −0.01 | 0.05 | −0.02 | 0.04 | −0.01 | −0.10 | −0.18 | −0.22 | 0.19 | 0.08 | 0.01 | −0.05 | 0.17 | 0.12 |
| E2F-High | −0.33 | −0.34 | −0.10 | −0.32 | −0.27 | −0.01 | 0.06 | 0.05 | 0.42 | 0.34 | 0.00 | 0.03 | −0.12 | 0.13 | 0.14 | −0.02 | −0.09 | 0.60 | 0.47 |

TABLE 5

| | Ha-ras | Ki-ras | N-ras | v-src | erbB2 | abl | Ras-N-17 | Rb | p53(wt) | Cip-1 | p16/INK4A | HSP40 | HSP90 | Hsdj | DnaJ-61 | Bax (N) | Bax (F) | IkB-TDN | IKK-DN |
|---|---|---|---|---|---|---|---|---|---|---|---|---|---|---|---|---|---|---|---|
| E2F-Low | 0.07 | −0.11 | 0.06 | −0.24 | −0.08 | −0.17 | 0.01 | 0.21 | 0.29 | −0.17 | 0.04 | −0.12 | −0.09 | −0.14 | 0.39 | −0.20 | −0.23 | 0.33 | 0.03 |
| FBRCA1-13 | −0.02 | −0.15 | −0.15 | 0.03 | −0.11 | −0.08 | −0.09 | 0.01 | −0.25 | −0.13 | −0.13 | 0.27 | −0.14 | 0.02 | 0.21 | −0.12 | −0.14 | −0.20 | −0.09 |
| FMDM2hwt-6 | 0.10 | 0.07 | 0.13 | 0.23 | −0.03 | −0.48 | −0.18 | −0.01 | −0.50 | −0.10 | −0.35 | 0.21 | −0.43 | −0.10 | −0.10 | −0.03 | −0.09 | −0.18 | −0.19 |
| p27-3 | −0.17 | −0.36 | −0.31 | −0.47 | −0.41 | −0.29 | −0.01 | 0.50 | 0.21 | −0.16 | 0.17 | 0.18 | 0.41 | −0.01 | 0.23 | 0.00 | 0.15 | 0.24 | 0.15 |
| CAPN10-10 | −0.02 | 0.05 | 0.01 | −0.17 | 0.12 | 0.21 | 0.21 | 0.18 | 0.56 | 0.12 | 0.23 | −0.26 | 0.45 | 0.13 | 0.28 | −0.20 | 0.04 | 0.21 | 0.06 |
| c-myc-1 | −0.17 | −0.26 | −0.22 | −0.30 | −0.26 | −0.16 | 0.10 | 0.15 | 0.51 | 0.16 | 0.21 | −0.03 | 0.37 | −0.06 | 0.04 | −0.07 | 0.13 | 0.24 | 0.10 |
| MSSP-10 | −0.02 | −0.14 | 0.17 | −0.21 | −0.18 | −0.47 | −0.25 | 0.23 | −0.33 | −0.26 | −0.19 | 0.19 | −0.19 | −0.10 | 0.14 | 0.04 | −0.06 | 0.11 | 0.01 |
| MM1 | −0.41 | −0.23 | −0.30 | −0.28 | −0.14 | 0.30 | 0.08 | 0.14 | 0.48 | 0.30 | 0.26 | 0.27 | 0.55 | 0.39 | 0.25 | 0.04 | 0.15 | 0.21 | 0.28 |
| AMY1 | 0.06 | 0.03 | 0.03 | −0.28 | 0.05 | 0.37 | 0.15 | 0.33 | 0.44 | −0.07 | 0.24 | −0.08 | 0.32 | 0.18 | 0.48 | 0.08 | −0.03 | 0.05 | 0.13 |
| Max | −0.04 | −0.16 | −0.08 | −0.29 | −0.15 | 0.27 | 0.00 | 0.32 | 0.21 | −0.06 | 0.19 | 0.10 | 0.29 | 0.08 | 0.22 | 0.02 | 0.01 | −0.11 | −0.03 |
| MDM2-hmut | −0.33 | −0.32 | −0.37 | −0.31 | −0.21 | 0.24 | 0.21 | 0.27 | 0.62 | 0.39 | 0.45 | 0.20 | 0.54 | 0.38 | 0.27 | 0.01 | 0.28 | 0.33 | 0.33 |
| MDM2-mWT | 0.04 | 0.02 | −0.05 | −0.02 | 0.02 | 0.19 | 0.26 | 0.15 | 0.48 | 0.04 | 0.33 | 0.01 | 0.24 | 0.04 | 0.20 | −0.29 | 0.03 | 0.19 | −0.04 |
| TERT-WT | 0.22 | 0.20 | −0.01 | 0.15 | −0.02 | 0.18 | −0.02 | −0.08 | 0.04 | −0.08 | −0.06 | −0.02 | −0.19 | −0.18 | −0.11 | −0.10 | 0.02 | −0.13 | −0.15 |
| TERT-DN | −0.32 | −0.13 | −0.14 | −0.17 | 0.01 | 0.54 | 0.23 | −0.02 | 0.44 | 0.36 | 0.34 | −0.15 | 0.53 | 0.34 | 0.25 | −0.06 | 0.07 | 0.12 | 0.24 |
| PTEN-WT | −0.07 | −0.11 | 0.01 | −0.31 | −0.07 | 0.32 | 0.12 | −0.16 | 0.22 | 0.03 | 0.21 | −0.16 | 0.24 | 0.06 | 0.39 | −0.01 | 0.10 | 0.03 | 0.11 |
| PTEN-A3 | −0.12 | −0.24 | 0.07 | −0.16 | 0.03 | 0.27 | −0.05 | −0.25 | −0.07 | −0.20 | 0.15 | −0.01 | −0.04 | −0.07 | 0.12 | 0.25 | −0.12 | −0.31 | −0.04 |
| PTEN-G129R | 0.15 | 0.00 | 0.13 | −0.27 | 0.08 | 0.29 | 0.37 | 0.09 | 0.41 | −0.12 | 0.18 | −0.25 | 0.16 | 0.04 | 0.51 | −0.30 | −0.15 | 0.10 | −0.11 |
| Bcl-2 | −0.26 | −0.30 | −0.33 | −0.24 | −0.30 | 0.27 | 0.06 | 0.26 | 0.12 | 0.15 | 0.21 | 0.23 | 0.22 | 0.08 | −0.09 | 0.13 | 0.12 | −0.07 | 0.16 |
| per2 | −0.11 | −0.06 | 0.06 | −0.31 | −0.05 | 0.34 | 0.20 | 0.14 | 0.35 | 0.23 | 0.10 | −0.09 | 0.29 | 0.34 | 0.18 | 0.28 | −0.04 | 0.14 | 0.29 |
| per3 | 0.17 | 0.12 | 0.22 | −0.17 | 0.09 | 0.11 | 0.08 | 0.14 | 0.16 | 0.03 | −0.11 | 0.00 | 0.14 | 0.24 | 0.10 | 0.17 | −0.02 | 0.16 | 0.18 |
| Cyclin D1-11 | 0.15 | 0.10 | 0.21 | 0.04 | 0.04 | −0.05 | −0.24 | 0.01 | −0.37 | −0.19 | −0.28 | 0.05 | −0.23 | 0.03 | 0.20 | −0.16 | −0.25 | −0.07 | −0.08 |
| STAT2-4 | 0.07 | 0.05 | 0.06 | −0.07 | −0.13 | −0.41 | −0.42 | 0.19 | −0.15 | −0.18 | −0.01 | −0.09 | 0.03 | −0.31 | −0.04 | 0.08 | 0.06 | −0.08 | −0.04 |
| TSC1-4 | −0.27 | −0.41 | −0.36 | −0.37 | −0.27 | 0.09 | −0.04 | 0.27 | 0.30 | 0.20 | 0.05 | 0.52 | 0.24 | 0.16 | −0.09 | 0.48 | 0.21 | 0.24 | 0.43 |
| Bad-22 | −0.11 | 0.02 | −0.05 | −0.10 | 0.05 | −0.10 | 0.01 | 0.33 | 0.35 | 0.36 | −0.15 | 0.30 | 0.10 | 0.23 | 0.06 | 0.16 | 0.16 | 0.46 | 0.39 |
| FAPP-4 | 0.14 | 0.17 | 0.20 | −0.05 | −0.08 | −0.11 | 0.04 | 0.19 | −0.10 | −0.15 | −0.26 | −0.17 | −0.18 | 0.04 | 0.07 | 0.02 | −0.23 | 0.22 | 0.05 |
| FHO-6 | −0.06 | −0.07 | 0.01 | −0.14 | −0.12 | −0.08 | 0.03 | 0.32 | 0.01 | 0.01 | −0.03 | 0.31 | 0.22 | 0.27 | 0.19 | 0.06 | 0.04 | 0.15 | 0.19 |
| F25 + lactacystin | −0.15 | −0.10 | −0.21 | −0.22 | −0.05 | 0.00 | 0.09 | 0.40 | 0.29 | 0.11 | 0.20 | 0.26 | 0.36 | 0.33 | 0.15 | 0.04 | 0.27 | 0.23 | 0.36 |
| F25 + ONO5046 | −0.15 | −0.18 | −0.27 | −0.08 | −0.21 | −0.49 | −0.23 | 0.41 | −0.08 | 0.08 | −0.04 | 0.29 | −0.01 | −0.17 | −0.30 | −0.03 | 0.37 | 0.05 | 0.10 |
| F25 + CA-074 | −0.01 | 0.03 | −0.13 | −0.07 | −0.09 | −0.46 | −0.34 | 0.47 | −0.08 | −0.10 | −0.15 | 0.32 | 0.04 | −0.04 | −0.03 | −0.07 | 0.17 | 0.11 | 0.08 |
| F25 + PQQ | 0.08 | 0.05 | −0.01 | −0.03 | −0.15 | −0.59 | −0.37 | 0.37 | −0.30 | −0.27 | −0.16 | 0.27 | −0.05 | −0.14 | −0.13 | −0.07 | 0.15 | −0.01 | −0.16 |
| F25 + PD98059 | 0.02 | 0.17 | 0.12 | 0.10 | 0.10 | −0.03 | 0.10 | 0.01 | 0.27 | −0.02 | 0.19 | −0.40 | 0.05 | −0.20 | −0.01 | −0.21 | 0.08 | 0.04 | −0.13 |
| F25 + ALLN | −0.24 | −0.08 | −0.13 | 0.05 | 0.01 | 0.17 | 0.15 | −0.14 | 0.26 | 0.23 | 0.26 | 0.06 | 0.10 | 0.08 | −0.03 | 0.14 | 0.22 | −0.10 | 0.15 |
| F25 + ONO3403 | −0.47 | −0.52 | −0.55 | −0.36 | −0.25 | 0.08 | −0.09 | 0.21 | 0.35 | 0.22 | 0.28 | 0.43 | 0.38 | 0.30 | 0.20 | 0.19 | 0.23 | 0.31 | 0.55 |
| F25 + Y27632 | −0.11 | −0.26 | −0.23 | −0.30 | 0.01 | −0.04 | 0.10 | 0.30 | 0.56 | 0.18 | 0.27 | 0.12 | 0.34 | 0.19 | 0.45 | −0.11 | −0.03 | 0.35 | 0.29 |

TABLE 6

| | PDGF-R | PDGF-R | Glucoc | RhoA | RhoA-D | CaMKII: | CaMKII | PKCalph | PKCalpt | PKCeps | PKCeps | TPA (PK | Akt-DN | ERK-DN | JNK-DI | p38-DN | cAMP |
|---|---|---|---|---|---|---|---|---|---|---|---|---|---|---|---|---|---|
| PDGF-R | 1.00 | | | | | | | | | | | | | | | | |
| PDGF-R-Del | 0.08 | 1.00 | | | | | | | | | | | | | | | |
| Glucocorticoid-R | 0.03 | 0.37 | 1.00 | | | | | | | | | | | | | | |
| RhoA | 0.16 | 0.31 | 0.32 | 1.00 | | | | | | | | | | | | | |
| RhoA-DN | 0.20 | 0.61 | −0.02 | 0.29 | 1.00 | | | | | | | | | | | | |
| CaMKIIa | −0.15 | −0.07 | 0.01 | −0.12 | 0.04 | 1.00 | | | | | | | | | | | |
| CaMKIIa-Active | 0.15 | 0.39 | 0.20 | 0.19 | 0.29 | 0.16 | 1.00 | | | | | | | | | | |
| PKCalpha-KN | 0.40 | −0.05 | 0.05 | 0.45 | 0.19 | −0.28 | −0.19 | 1.00 | | | | | | | | | |
| PKCalpha-KN | 0.11 | 0.40 | 0.09 | 0.24 | 0.45 | 0.03 | 0.02 | 0.21 | 1.00 | | | | | | | | |
| PKCepsilon-KN | 0.00 | 0.25 | 0.12 | 0.41 | 0.25 | −0.06 | −0.14 | 0.49 | 0.13 | 1.00 | | | | | | | |
| PKCepsilon-KN | 0.20 | 0.10 | 0.12 | 0.03 | 0.02 | −0.08 | −0.21 | 0.07 | 0.69 | 0.01 | 1.00 | | | | | | |
| TPA (PKC−) | −0.18 | 0.20 | 0.11 | −0.15 | −0.03 | −0.11 | 0.11 | 0.15 | 0.09 | 0.24 | −0.20 | 1.00 | | | | | |
| Akt-DN | 0.49 | −0.10 | −0.08 | 0.00 | −0.22 | −0.02 | −0.16 | −0.01 | −0.17 | 0.44 | 0.00 | 1.00 | | | | | |
| ERK-DN | −0.06 | 0.62 | 0.09 | 0.32 | 0.72 | 0.16 | 0.39 | 0.16 | 0.35 | −0.14 | 0.11 | −0.04 | 1.00 | | | | |
| JNK-DN | −0.12 | 0.53 | 0.14 | 0.10 | 0.28 | 0.12 | −0.10 | 0.12 | 0.10 | 0.19 | 0.30 | 0.16 | 0.37 | 1.00 | | | |
| p38-DN | −0.11 | 0.37 | −0.13 | 0.05 | 0.56 | 0.15 | 0.17 | 0.12 | 0.09 | 0.17 | 0.16 | 0.15 | 0.63 | 0.54 | 1.00 | | |
| cAMP-PK-CS | 0.18 | 0.36 | 0.40 | 0.30 | −0.15 | −0.06 | 0.21 | 0.03 | 0.40 | 0.30 | 0.34 | 0.47 | −0.01 | 0.06 | 0.13 | −0.07 | 1.00 |

TABLE 7

| | PDGF- | PDGF- | | | RhoA- | | CaMKlla- | | | | | TPA | Akt- | ERK- | JNK- | p38- | cAMP- | | | caspase |
|---|---|---|---|---|---|---|---|---|---|---|---|---|---|---|---|---|---|---|---|---|
| | R | R-D [symbol] | Glucocor | RhoA | DN | CaMKlla | Ac | PKCalpha | PKCalpha | PKCepsil [symbol] | PKCepsil [symbol] | (PKC-) | DN | DN | DN | DN | PK- [symbol] | 14-3-3zW | TK | -1 |
| 14-3-3zWT | −0.12 | 0.08 | −0.02 | −0.17 | 0.08 | 0.13 | 0.00 | 0.11 | 0.19 | 0.44 | 0.07 | 0.33 | 0.13 | 0.21 | 0.09 | 0.17 | 0.13 | 1.00 | | |
| TK | 0.02 | −0.17 | −0.06 | −0.07 | −0.28 | 0.11 | −0.13 | −0.27 | −0.07 | −0.21 | −0.06 | −0.26 | 0.08 | −0.26 | −0.23 | −0.22 | −0.27 | −0.02 | 1.00 | |
| caspase-1 | 0.07 | 0.18 | 0.07 | 0.05 | 0.16 | 0.17 | 0.41 | −0.18 | −0.30 | −0.15 | −0.46 | 0.10 | −0.17 | 0.33 | 0.01 | 0.12 | −0.09 | −0.30 | −0.02 | 1.00 |
| caspase-3 | 0.01 | 0.48 | 0.38 | 0.57 | 0.30 | −0.14 | 0.32 | 0.20 | 0.06 | 0.34 | −0.31 | 0.21 | −0.12 | 0.60 | 0.15 | 0.21 | 0.19 | 0.01 | −0.02 | 0.49 |
| caspase-2 | 0.13 | 0.37 | 0.06 | 0.09 | 0.44 | −0.31 | −0.12 | 0.44 | 0.21 | 0.24 | −0.03 | 0.19 | −0.03 | 0.41 | 0.27 | 0.30 | −0.01 | 0.20 | −0.25 | −0.09 |
| cystatin alpha | 0.24 | 0.42 | 0.19 | 0.54 | 0.35 | 0.05 | 0.43 | 0.05 | 0.23 | −0.15 | −0.05 | 0.05 | 0.23 | 0.46 | 0.15 | 0.26 | 0.29 | −0.14 | −0.12 | 0.69 |
| cystatin E | 0.38 | 0.16 | −0.01 | 0.25 | 0.18 | −0.01 | 0.24 | 0.03 | 0.16 | 0.02 | 0.16 | 0.01 | 0.57 | 0.41 | 0.13 | 0.27 | 0.02 | 0.23 | 0.19 | 0.20 |
| u-calpain | 0.05 | 0.48 | 0.21 | 0.11 | 0.49 | 0.04 | 0.12 | −0.03 | 0.11 | −0.13 | −0.10 | −0.03 | 0.23 | 0.35 | 0.48 | 0.41 | −0.16 | 0.04 | −0.16 | 0.18 |
| m-calpain | 0.13 | 0.54 | 0.44 | 0.57 | 0.36 | −0.15 | 0.15 | 0.13 | 0.38 | 0.31 | 0.00 | 0.16 | −0.15 | 0.23 | 0.24 | 0.15 | 0.43 | 0.10 | −0.25 | 0.06 |
| calpain 30K | 0.12 | −0.29 | −0.02 | −0.26 | −0.01 | −0.35 | −0.37 | 0.38 | 0.11 | −0.06 | 0.24 | −0.02 | 0.13 | −0.50 | −0.19 | −0.23 | −0.10 | 0.05 | −0.08 | −0.48 |
| (N) | | | | | | | | | | | | | | | | | | | | |
| calpain 30K (F) | 0.14 | 0.19 | −0.08 | −0.12 | 0.28 | 0.07 | 0.19 | −0.12 | −0.26 | −0.04 | −0.44 | −0.01 | 0.24 | 0.20 | 0.10 | 0.03 | −0.11 | 0.21 | 0.14 | 0.15 |
| calpastatin | 0.02 | −0.28 | −0.10 | −0.27 | −0.27 | 0.19 | −0.38 | −0.02 | 0.25 | −0.09 | 0.44 | −0.29 | 0.00 | −0.49 | −0.25 | −0.19 | −0.11 | 0.08 | 0.32 | −0.34 |
| CP-antisense | −0.10 | 0.30 | −0.12 | 0.17 | 0.53 | −0.01 | 0.15 | 0.17 | 0.35 | 0.31 | 0.06 | 0.08 | 0.11 | 0.68 | 0.06 | 0.60 | 0.03 | 0.17 | −0.16 | 0.16 |
| BH | −0.16 | −0.04 | −0.04 | −0.12 | 0.18 | 0.35 | 0.05 | −0.13 | −0.03 | −0.02 | 0.05 | 0.05 | 0.26 | −0.01 | 0.15 | 0.25 | −0.26 | 0.23 | 0.17 | −0.23 |
| DAN | 0.20 | 0.37 | 0.15 | 0.34 | 0.40 | −0.18 | 0.05 | 0.28 | 0.03 | 0.30 | −0.21 | 0.19 | −0.01 | 0.47 | 0.22 | 0.23 | 0.16 | 0.16 | −0.06 | 0.22 |
| Regucalcin | 0.00 | 0.19 | −0.01 | 0.04 | 0.25 | 0.18 | 0.05 | −0.15 | 0.72 | 0.06 | 0.65 | 0.11 | 0.35 | 0.02 | 0.24 | 0.34 | 0.14 | 0.25 | −0.06 | −0.36 |
| CathL-mut | −0.33 | −0.08 | −0.21 | −0.17 | −0.01 | 0.32 | −0.25 | 0.10 | −0.01 | 0.46 | −0.10 | 0.11 | −0.21 | 0.00 | 0.07 | 0.11 | −0.02 | 0.43 | 0.08 | −0.26 |
| STAT1 | −0.01 | 0.44 | 0.78 | 0.34 | −0.18 | 0.05 | 0.28 | −0.15 | 0.01 | 0.12 | 0.04 | 0.23 | 0.12 | 0.11 | 0.29 | −0.05 | 0.52 | 0.09 | 0.07 | 0.07 |
| CBP | −0.10 | 0.47 | 0.21 | 0.18 | 0.61 | 0.30 | 0.50 | −0.26 | 0.03 | 0.22 | −0.14 | −0.14 | −0.25 | 0.56 | −0.03 | 0.17 | −0.01 | 0.12 | −0.05 | 0.34 |
| P/CAF | 0.16 | 0.21 | −0.05 | 0.11 | 0.36 | −0.03 | −0.03 | 0.02 | 0.13 | 0.03 | −0.21 | 0.02 | −0.14 | 0.16 | 0.00 | −0.14 | −0.13 | 0.06 | 0.25 | −0.01 |
| HNF1 | 0.05 | 0.33 | 0.33 | 0.36 | −0.13 | 0.07 | 0.29 | −0.22 | 0.12 | 0.08 | −0.03 | 0.06 | 0.25 | 0.17 | 0.07 | −0.06 | 0.32 | 0.15 | 0.17 | 0.09 |
| HNF3b | −0.16 | −0.03 | −0.04 | 0.33 | 0.10 | −0.07 | −0.39 | 0.43 | 0.38 | 0.51 | 0.26 | 0.02 | 0.21 | 0.07 | 0.16 | 0.25 | −0.08 | 0.38 | 0.19 | −0.31 |
| HNF4 | −0.45 | −0.06 | −0.06 | −0.15 | −0.09 | 0.38 | −0.06 | −0.16 | 0.04 | −0.09 | −0.01 | 0.09 | 0.15 | 0.11 | 0.26 | 0.13 | −0.05 | 0.24 | 0.02 | −0.21 |
| coup | −0.18 | 0.52 | −0.01 | 0.29 | 0.36 | 0.18 | 0.56 | −0.18 | 0.20 | 0.19 | −0.17 | 0.10 | 0.05 | 0.64 | 0.12 | 0.42 | 0.24 | 0.22 | −0.04 | 0.19 |
| C/EBPa | 0.03 | 0.19 | 0.06 | 0.31 | 0.24 | −0.20 | 0.12 | 0.09 | 0.26 | −0.06 | −0.07 | 0.02 | −0.08 | 0.27 | −0.02 | 0.09 | 0.02 | −0.05 | 0.37 | 0.22 |
| C/EBPb | 0.24 | 0.39 | 0.15 | 0.11 | 0.30 | −0.42 | 0.20 | 0.17 | 0.09 | 0.22 | −0.09 | 0.05 | −0.01 | 0.47 | 0.04 | 0.05 | 0.13 | 0.12 | 0.01 | −0.04 |
| per-1 | 0.21 | 0.02 | 0.02 | 0.04 | 0.15 | 0.01 | −0.23 | 0.39 | 0.45 | 0.04 | 0.30 | 0.24 | 0.39 | 0.14 | 0.47 | 0.45 | −0.04 | 0.27 | −0.18 | −0.13 |
| p33/ING1 | 0.00 | 0.30 | −0.08 | 0.13 | 0.26 | −0.01 | 0.35 | −0.30 | −0.03 | −0.03 | −0.20 | −0.08 | −0.30 | 0.13 | −0.10 | −0.08 | 0.11 | −0.12 | 0.16 | 0.03 |
| T.Tn | 0.11 | −0.23 | 0.06 | −0.11 | −0.26 | 0.15 | 0.06 | −0.25 | −0.02 | −0.48 | 0.02 | −0.31 | 0.05 | −0.29 | −0.20 | −0.22 | −0.06 | −0.18 | 0.40 | 0.07 |
| CD44/H-CAM | 0.15 | 0.15 | −0.10 | −0.13 | 0.20 | 0.07 | 0.03 | −0.15 | 0.03 | −0.06 | 0.21 | −0.09 | 0.15 | 0.07 | 0.07 | 0.07 | 0.27 | 0.07 | −0.12 | −0.22 |
| CD44/3E | 0.38 | 0.16 | −0.01 | −0.38 | 0.34 | −0.19 | 0.01 | −0.27 | −0.30 | −0.19 | 0.00 | −0.19 | −0.09 | −0.07 | −0.19 | −0.21 | −0.19 | −0.07 | 0.01 | −0.08 |
| CD44/3s | 0.05 | 0.04 | −0.05 | −0.15 | 0.35 | −0.08 | −0.26 | −0.07 | −0.31 | 0.07 | −0.31 | −0.18 | −0.12 | −0.01 | −0.24 | −0.18 | −0.21 | 0.05 | 0.11 | −0.15 |
| Jagged-1 | −0.06 | −0.01 | 0.04 | 0.13 | 0.09 | 0.14 | 0.31 | −0.05 | −0.17 | −0.08 | −0.18 | −0.05 | −0.29 | 0.00 | −0.11 | 0.07 | −0.07 | −0.09 | −0.06 | 0.26 |
| p94-WT | −0.41 | 0.47 | 0.02 | 0.10 | 0.25 | −0.20 | 0.18 | −0.13 | −0.02 | 0.18 | −0.29 | 0.23 | −0.25 | 0.19 | 0.27 | 0.17 | −0.07 | 0.32 | −0.07 | −0.06 |
| p94-mut | −0.28 | 0.26 | 0.07 | −0.02 | −0.24 | −0.23 | −0.15 | −0.05 | −0.05 | 0.07 | −0.09 | 0.23 | 0.13 | −0.11 | 0.44 | 0.02 | 0.12 | 0.15 | −0.17 | −0.29 |
| E2F-High | 0.03 | 0.12 | 0.04 | −0.07 | 0.03 | −0.10 | −0.12 | −0.17 | −0.14 | 0.19 | −0.13 | 0.09 | 0.32 | −0.03 | 0.11 | 0.03 | −0.01 | 0.41 | −0.08 | −0.16 |
| 14-3-3zWT | −0.12 | 0.08 | −0.02 | −0.17 | 0.08 | 0.13 | 0.00 | 0.11 | 0.19 | 0.44 | 0.07 | 0.33 | 0.13 | 0.21 | 0.09 | 0.17 | 0.13 | 1.00 | | |

TABLE 8

| PDGF- | PDGF- | | | RhoA- | | CaMKlla- | | | | | TPA | Akt- | ERK- | JNK- | p38- | cAMP- | | | caspase- |
|---|---|---|---|---|---|---|---|---|---|---|---|---|---|---|---|---|---|---|---|
| R | R-D [symbol] | Glucocor | RhoA | DN | CaMKlla | Ac | PKCalpha | PKCalpha | PKCepsil [symbol] | PKCepsil [symbol] | (PKC-) | DN | DN | DN | DN | PK- [symbol] | 14-3-3zW | TK | 1 |

| | | | | | | | | | | | | | | | | | | | | |
|---|---|---|---|---|---|---|---|---|---|---|---|---|---|---|---|---|---|---|---|---|
| E2F-Low | −0.07 | −0.19 | 0.05 | −0.10 | −0.25 | 0.10 | −0.16 | −0.33 | −0.22 | 0.17 | −0.01 | −0.05 | 0.30 | −0.18 | −0.38 | −0.32 | −0.02 | 0.34 | 0.18 | −0.04 |
| FBRCA1-13 | 0.00 | −0.29 | −0.16 | 0.09 | 0.03 | 0.07 | −0.08 | 0.24 | −0.11 | 0.02 | 0.02 | −0.17 | −0.09 | −0.04 | −0.17 | −0.01 | −0.23 | −0.05 | −0.25 | −0.15 |
| FMDM2hwt-6 | −0.04 | −0.31 | −0.12 | −0.09 | −0.01 | 0.15 | −0.08 | 0.10 | −0.08 | −0.19 | 0.16 | −0.16 | −0.12 | −0.31 | 0.03 | 0.08 | −0.35 | −0.01 | −0.19 | −0.08 |
| p27-3 | −0.49 | 0.23 | 0.08 | 0.27 | 0.19 | 0.24 | 0.18 | −0.08 | −0.21 | 0.30 | −0.20 | −0.06 | −0.15 | 0.34 | 0.09 | 0.13 | −0.05 | 0.20 | −0.05 | 0.04 |
| CAPN10-10 | −0.06 | 0.47 | 0.25 | 0.13 | −0.13 | −0.09 | 0.19 | −0.32 | 0.07 | −0.05 | −0.08 | 0.10 | 0.23 | 0.30 | 0.25 | 0.01 | 0.31 | 0.03 | 0.31 | 0.23 |
| c-myc-1 | −0.21 | 0.37 | 0.18 | −0.03 | −0.08 | −0.04 | 0.17 | −0.17 | 0.04 | −0.01 | 0.04 | 0.26 | 0.22 | 0.28 | 0.41 | 0.32 | 0.23 | 0.22 | −0.08 | 0.15 |
| MSSP-10 | −0.23 | −0.41 | −0.17 | −0.18 | 0.02 | 0.28 | 0.14 | −0.15 | −0.26 | 0.01 | −0.07 | −0.16 | −0.10 | −0.17 | −0.31 | 0.02 | −0.34 | 0.18 | −0.13 | −0.01 |
| MM1 | 0.28 | 0.18 | −0.15 | 0.26 | 0.29 | −0.26 | 0.27 | 0.15 | −0.05 | 0.14 | −0.08 | 0.33 | 0.20 | 0.32 | 0.13 | 0.10 | 0.20 | 0.16 | −0.31 | 0.11 |
| AMY1 | −0.18 | 0.28 | 0.25 | 0.15 | −0.05 | 0.00 | 0.11 | −0.14 | 0.09 | 0.25 | 0.00 | 0.19 | −0.06 | 0.18 | −0.20 | −0.09 | 0.36 | 0.21 | 0.21 | 0.03 |
| Max | 0.00 | 0.21 | 0.12 | 0.24 | 0.04 | −0.03 | 0.10 | 0.01 | −0.07 | 0.21 | −0.11 | 0.03 | −0.19 | 0.26 | −0.21 | −0.13 | 0.25 | 0.02 | 0.24 | 0.18 |
| MDM2-hmut | −0.16 | 0.48 | 0.23 | 0.34 | 0.33 | −0.17 | 0.31 | 0.00 | 0.11 | 0.23 | −0.27 | 0.36 | 0.17 | 0.53 | 0.08 | 0.24 | 0.30 | 0.20 | −0.01 | 0.18 |
| MDM2-mWT | 0.08 | 0.02 | 0.05 | −0.01 | −0.19 | −0.19 | 0.12 | −0.10 | 0.06 | 0.07 | 0.03 | 0.26 | 0.52 | 0.07 | −0.16 | 0.07 | 0.28 | 0.31 | −0.06 | 0.02 |
| TERT-WT | 0.02 | −0.09 | −0.06 | −0.05 | −0.32 | −0.22 | −0.34 | 0.19 | −0.11 | 0.15 | 0.02 | 0.11 | 0.00 | −0.15 | −0.13 | −0.03 | 0.23 | 0.00 | −0.10 | −0.15 |
| TERT-DN | 0.45 | 0.30 | 0.11 | 0.21 | 0.14 | −0.30 | 0.18 | 0.08 | −0.06 | 0.00 | −0.19 | 0.02 | 0.11 | 0.29 | 0.03 | −0.15 | 0.24 | −0.09 | 0.12 | 0.21 |
| PTEN-WT | 0.05 | 0.28 | 0.39 | 0.14 | 0.05 | −0.04 | 0.15 | −0.07 | −0.14 | 0.16 | −0.15 | 0.08 | −0.09 | 0.17 | 0.01 | −0.16 | 0.25 | −0.06 | −0.11 | 0.32 |
| PTEN-A3 | −0.32 | 0.08 | 0.01 | 0.14 | −0.01 | 0.10 | 0.03 | −0.11 | 0.08 | 0.17 | −0.06 | 0.12 | −0.24 | 0.17 | 0.16 | 0.02 | 0.13 | −0.11 | −0.06 | 0.26 |
| PTEN-G129R | −0.01 | 0.10 | 0.37 | 0.24 | −0.27 | 0.00 | 0.07 | −0.16 | −0.05 | −0.01 | −0.08 | −0.02 | 0.26 | 0.00 | −0.08 | −0.34 | 0.18 | 0.01 | 0.21 | 0.15 |
| Bcl-2 | −0.10 | 0.19 | −0.03 | 0.03 | 0.19 | 0.02 | 0.00 | 0.05 | 0.01 | 0.20 | −0.20 | 0.20 | −0.16 | 0.27 | 0.02 | 0.15 | 0.18 | 0.01 | −0.02 | 0.02 |
| per2 | 0.18 | 0.47 | 0.27 | 0.13 | 0.15 | 0.08 | 0.19 | −0.14 | 0.27 | −0.02 | 0.13 | 0.24 | 0.03 | 0.19 | 0.25 | −0.02 | 0.40 | −0.01 | 0.16 | 0.05 |
| per3 | 0.15 | 0.31 | 0.42 | 0.31 | 0.04 | 0.23 | 0.21 | −0.06 | 0.37 | −0.09 | 0.22 | 0.25 | −0.02 | −0.03 | 0.21 | −0.08 | 0.38 | 0.04 | 0.17 | −0.05 |
| Cyclin D1-11 | 0.27 | −0.18 | 0.05 | 0.11 | −0.09 | 0.21 | 0.04 | −0.23 | −0.24 | −0.18 | 0.01 | −0.23 | −0.15 | −0.36 | −0.30 | −0.39 | −0.07 | −0.11 | 0.16 | −0.10 |
| STAT2-4 | −0.10 | 0.14 | 0.29 | −0.06 | −0.19 | 0.31 | 0.13 | −0.14 | 0.09 | 0.10 | 0.28 | 0.12 | −0.37 | −0.08 | 0.15 | 0.01 | 0.41 | 0.11 | −0.12 | −0.12 |
| TSC1-4 | −0.36 | 0.17 | 0.03 | 0.27 | 0.33 | 0.15 | −0.04 | 0.32 | 0.25 | 0.54 | −0.01 | 0.36 | −0.10 | 0.36 | 0.32 | 0.37 | 0.29 | 0.32 | −0.25 | −0.15 |
| Bad-22 | 0.13 | 0.24 | 0.04 | 0.37 | 0.35 | 0.11 | 0.00 | 0.09 | 0.25 | 0.14 | −0.03 | −0.26 | −0.02 | 0.13 | 0.17 | 0.14 | −0.04 | 0.07 | 0.19 | 0.00 |
| FAPP-4 | −0.06 | −0.26 | −0.15 | −0.34 | −0.24 | 0.22 | −0.25 | −0.20 | −0.28 | 0.06 | 0.03 | −0.03 | 0.04 | −0.28 | −0.09 | −0.24 | 0.01 | 0.25 | 0.10 | −0.21 |
| FHO-6 | −0.20 | 0.48 | 0.26 | 0.25 | 0.34 | 0.34 | 0.41 | −0.01 | 0.23 | 0.24 | 0.05 | 0.28 | −0.23 | 0.31 | 0.43 | 0.23 | 0.26 | 0.26 | −0.20 | 0.05 |
| F25 + | −0.09 | 0.44 | 0.09 | 0.31 | 0.40 | 0.04 | 0.33 | 0.13 | 0.23 | 0.17 | −0.1.5 | 0.31 | −0.22 | 0.29 | 0.21 | 0.20 | 0.21 | 0.37 | −0.21 | 0.05 |
| lactacystin | | | | | | | | | | | | | | | | | | | | |
| F25 + | −0.46 | 0.10 | −0.09 | 0.05 | 0.16 | 0.17 | 0.09 | 0.02 | −0.07 | 0.09 | −0.16 | 0.15 | −0.28 | 0.15 | 0.11 | 0.29 | 0.03 | 0.17 | −0.07 | −0.13 |
| ONO5046 | | | | | | | | | | | | | | | | | | | | |
| F25 + | −0.30 | 0.06 | −0.17 | 0.04 | 0.14 | 0.30 | 0.12 | −0.18 | −0.01 | 0.09 | −0.07 | 0.12 | −0.28 | 0.03 | −0.05 | 0.11 | 0.12 | 0.19 | −0.08 | −0.04 |
| CA-074 | | | | | | | | | | | | | | | | | | | | |
| F25 + PQQ | −0.39 | −0.09 | −0.18 | −0.01 | 0.05 | 0.34 | 0.22 | −0.15 | −0.16 | −0.02 | −0.06 | 0.00 | −0.30 | −0.07 | −0.08 | 0.08 | −0.02 | 0.03 | −0.12 | 0.00 |
| F25 + | −0.06 | 0.29 | 0.09 | 0.08 | −0.32 | −0.23 | 0.05 | −0.29 | 0.05 | −0.09 | −0.01 | −0.03 | 0.12 | 0.04 | 0.14 | −0.05 | 0.22 | 0.03 | 0.22 | 0.05 |
| PD98059 | | | | | | | | | | | | | | | | | | | | |
| F25 + ALLN | −0.03 | 0.33 | −0.11 | 0.16 | 0.15 | −0.25 | −0.02 | 0.25 | 0.41 | 0.11 | 0.16 | 0.26 | 0.00 | 0.37 | 0.52 | 0.41 | 0.17 | 0.07 | −0.17 | 0.09 |
| F25 + | −0.45 | 0.50 | 0.12 | 0.25 | 0.52 | 0.10 | 0.19 | 0.02 | 0.33 | 0.41 | −0.13 | 0.30 | −0.16 | 0.53 | 0.30 | 0.51 | 0.22 | 0.38 | −0.24 | 0.03 |
| ONO3403 | | | | | | | | | | | | | | | | | | | | |
| F25 + Y27632 | −0.08 | 0.43 | 0.20 | 0.16 | 0.23 | 0.24 | 0.24 | −0.17 | 0.41 | 0.07 | 0.11 | 0.14 | 0.28 | 0.37 | 0.21 | 0.23 | 0.18 | 0.29 | 0.10 | 0.06 |

TABLE 9

| | | | | | | | calpain | calpain | | CP- | | | | | | | | |
|---|---|---|---|---|---|---|---|---|---|---|---|---|---|---|---|---|---|---|
| | caspase-3 | caspase-2 | cystatin al | cystatin E | u-calpain | m-calpain | 30K | 30K | calpastatin | antisens [symbol] | BH | DAN | Regucalci | CathL-mut | STAT1 | CBP | P/CAF | HNF1 |
| caspase-3 | 1.00 | | | | | | | | | | | | | | | | | |
| caspase-2 | 0.41 | 1.00 | | | | | | | | | | | | | | | | |
| cystatin alpha | 0.56 | 0.09 | 1.00 | | | | | | | | | | | | | | | |
| cystatin E | 0.32 | 0.33 | 0.54 | 1.00 | | | | | | | | | | | | | | |
| u-calpain | 0.25 | 0.31 | 0.37 | 0.27 | 1.00 | | | | | | | | | | | | | |
| m-calpain | 0.45 | 0.23 | 0.52 | 0.08 | 0.41 | 1.00 | | | | | | | | | | | | |
| calpain 30K (N) | −0.43 | 0.03 | −0.40 | −0.37 | −0.16 | −0.25 | 1.00 | | | | | | | | | | | |
| calpain 30K (F) | 0.12 | 0.18 | 0.21 | 0.43 | 0.55 | 0.14 | 0.13 | 1.00 | | | | | | | | | | |
| calpastatin | −0.53 | −0.17 | −0.35 | −0.21 | −0.18 | −0.23 | 0.14 | −0.20 | 1.00 | | | | | | | | | |
| CP-antisense | 0.38 | 0.25 | 0.28 | 0.31 | 0.09 | −0.02 | −0.28 | 0.01 | −0.19 | 1.00 | | | | | | | | |
| BH | −0.18 | 0.08 | −0.24 | 0.30 | 0.31 | −0.12 | −0.26 | 0.23 | 0.13 | 0.04 | 1.00 | | | | | | | |
| DAN | 0.68 | 0.69 | 0.54 | 0.45 | 0.33 | 0.35 | −0.37 | 0.32 | −0.36 | 0.35 | −0.03 | 1.00 | | | | | | |
| Regucalcin | −0.16 | 0.02 | −0.04 | 0.21 | 0.37 | 0.24 | 0.17 | 0.14 | 0.16 | 0.13 | 0.47 | −0.05 | 1.00 | | | | | |
| CathL-mut | −0.20 | −0.04 | −0.39 | 0.01 | −0.13 | 0.01 | −0.09 | 0.00 | 0.31 | 0.11 | 0.27 | −0.13 | 0.20 | 1.00 | | | | |
| STAT1 | 0.40 | −0.01 | 0.22 | 0.23 | 0.20 | 0.37 | −0.10 | 0.11 | −0.20 | −0.17 | 0.05 | 0.13 | 0.08 | −0.14 | 1.00 | | | |
| CBP | 0.41 | 0.10 | 0.32 | 0.32 | 0.16 | 0.17 | −0.42 | 0.24 | −0.35 | 0.42 | 0.19 | 0.26 | 0.14 | 0.13 | 0.17 | 1.00 | | |
| P/CAF | 0.21 | 0.27 | 0.02 | 0.04 | 0.28 | 0.32 | 0.32 | 0.34 | −0.10 | −0.11 | −0.07 | 0.33 | 0.05 | −0.15 | −0.04 | 0.05 | 1.00 | |
| HNF1 | 0.44 | −0.10 | 0.34 | 0.20 | 0.24 | 0.40 | −0.42 | 0.13 | −0.21 | 0.02 | 0.03 | 0.28 | 0.20 | −0.14 | 0.44 | 0.16 | 0.15 | 1.00 |
| HNF3b | 0.06 | 0.13 | −0.11 | 0.17 | −0.01 | 0.02 | 0.27 | −0.01 | 0.22 | 0.29 | 0.17 | 0.17 | 0.30 | 0.40 | −0.08 | −0.11 | −0.02 | 0.16 |
| HNF4 | −0.15 | −0.05 | −0.09 | −0.08 | 0.04 | −0.15 | −0.37 | 0.01 | −0.05 | 0.09 | 0.33 | −0.01 | 0.10 | 0.17 | −0.11 | 0.04 | −0.16 | 0.22 |
| coup | 0.42 | 0.06 | 0.35 | 0.35 | 0.31 | 0.34 | −0.66 | 0.20 | −0.24 | 0.54 | 0.21 | 0.26 | 0.22 | 0.16 | 0.15 | 0.45 | 0.11 | 0.53 |
| C/EBPa | 0.50 | 0.27 | 0.51 | 0.40 | 0.21 | 0.04 | −0.26 | 0.19 | −0.14 | 0.31 | −0.10 | 0.67 | −0.08 | −0.30 | 0.02 | 0.08 | 0.39 | 0.20 |
| C/EBPb | 0.40 | 0.22 | −0.07 | 0.02 | 0.17 | 0.20 | 0.02 | 0.14 | −0.40 | 0.30 | −0.28 | 0.36 | −0.15 | −0.17 | 0.08 | 0.11 | 0.26 | 0.34 |
| per-1 | −0.06 | 0.35 | 0.13 | 0.25 | 0.27 | 0.06 | 0.24 | 0.14 | −0.07 | 0.09 | 0.05 | 0.17 | 0.33 | 0.01 | −0.03 | −0.24 | −0.02 | −0.06 |
| p33/ING1 | 0.12 | −0.15 | −0.03 | −0.21 | 0.17 | 0.23 | 0.10 | 0.22 | −0.31 | −0.01 | −0.20 | 0.10 | 0.09 | −0.08 | 0.08 | 0.32 | 0.50 | 0.25 |
| T.Tn | −0.24 | −0.39 | 0.09 | −0.07 | −0.10 | −0.16 | −0.07 | 0.00 | −0.19 | −0.10 | −0.18 | −0.14 | −0.26 | −0.10 | −0.16 | −0.06 | 0.18 | |
| CD44/H-CAM | −0.21 | −0.06 | −0.20 | −0.12 | 0.14 | 0.01 | 0.34 | 0.24 | −0.07 | 0.16 | −0.06 | 0.09 | 0.12 | 0.05 | 0.08 | 0.18 | −0.05 | −0.15 |
| CD44/3E | −0.16 | 0.21 | −0.29 | 0.04 | 0.09 | −0.17 | 0.31 | 0.32 | −0.05 | −0.08 | 0.13 | 0.14 | 0.01 | −0.35 | −0.11 | 0.38 | 0.19 | −0.17 |
| CD44/3s | −0.03 | 0.10 | −0.25 | −0.06 | 0.17 | −0.02 | 0.43 | 0.45 | −0.08 | 0.06 | 0.11 | 0.19 | 0.04 | 0.05 | −0.14 | 0.32 | 0.32 | −0.10 |
| Jagged-1 | −0.03 | −0.15 | 0.25 | 0.10 | −0.01 | −0.11 | −0.37 | 0.04 | 0.09 | 0.06 | 0.18 | −0.06 | −0.08 | 0.29 | −0.01 | 0.41 | −0.39 | −0.13 |
| p94-WT | 0.29 | 0.24 | −0.17 | −0.09 | 0.36 | 0.41 | −0.11 | 0.30 | −0.12 | −0.06 | 0.15 | 0.08 | 0.10 | 0.16 | 0.15 | 0.16 | 0.25 | 0.30 |
| p94-mut | −0.06 | 0.08 | −0.21 | −0.06 | 0.22 | 0.25 | 0.02 | 0.13 | −0.03 | −0.33 | 0.09 | −0.17 | 0.07 | 0.16 | 0.30 | −0.34 | −0.02 | 0.11 |
| E2F-High | −0.02 | 0.09 | 0.03 | 0.26 | 0.46 | 0.30 | 0.15 | 0.50 | −0.07 | −0.23 | 0.35 | 0.12 | 0.37 | 0.12 | 0.20 | −0.03 | 0.15 | 0.33 |

TABLE 10

| | caspase-3 | caspase-2 | cystatin al | cystatin E | u-calpain | m-calpain | calpain 30K | calpain 30K | calpastatin | CP-antisens [symbol] | BH | DAN | Regucalci | CathL-mut | STAT1 | CBP | P/CAF | HNF1 |
|---|---|---|---|---|---|---|---|---|---|---|---|---|---|---|---|---|---|---|
| E2F-Low | −0.04 | −0.19 | −0.07 | 0.20 | 0.06 | −0.04 | −0.06 | 0.27 | 0.19 | 0.01 | 0.24 | 0.02 | 0.27 | 0.17 | 0.09 | 0.14 | −0.09 | 0.41 |
| FBRCA1-13 | −0.04 | 0.02 | −0.30 | −0.14 | 0.01 | −0.15 | 0.16 | 0.03 | −0.01 | 0.09 | −0.06 | 0.01 | 0.01 | −0.14 | −0.31 | −0.03 | −0.05 | −0.09 |
| FMDM2hwt- | −0.39 | −0.13 | −0.19 | −0.12 | 0.04 | −0.24 | 0.07 | −0.08 | 0.38 | −0.15 | 0.19 | −0.31 | 0.21 | 0.15 | −0.26 | −0.12 | −0.33 | −0.40 |
| 6 | | | | | | | | | | | | | | | | | | |
| p27-3 | 0.29 | 0.05 | −0.13 | 0.07 | 0.13 | 0.09 | −0.51 | 0.18 | −0.06 | 0.33 | 0.23 | 0.26 | −0.03 | 0.25 | 0.11 | 0.48 | −0.09 | 0.33 |
| CAPN10-10 | 0.44 | −0.01 | 0.37 | 0.32 | 0.21 | 0.18 | −0.30 | 0.24 | −0.28 | 0.07 | −0.14 | 0.25 | −0.10 | −0.16 | 0.52 | 0.11 | 0.18 | 0.56 |
| c-myc-1 | 0.26 | 0.13 | 0.25 | 0.36 | 0.38 | 0.27 | −0.55 | 0.15 | −0.29 | 0.10 | 0.09 | 0.20 | 0.03 | 0.15 | 0.35 | 0.10 | −0.18 | 0.40 |
| MSSP-10 | −0.26 | −0.23 | −0.21 | 0.01 | 0.03 | −0.22 | −0.02 | 0.10 | 0.35 | 0.01 | 0.35 | −0.21 | 0.23 | 0.29 | −0.23 | 0.13 | −0.24 | −0.21 |
| MM1 | 0.32 | 0.17 | 0.30 | 0.41 | 0.33 | 0.27 | −0.25 | 0.26 | −0.57 | 0.22 | 0.01 | 0.47 | 0.08 | −0.14 | −0.01 | 0.13 | 0.19 | 0.32 |
| AMY1 | 0.35 | −0.08 | 0.16 | 0.16 | −0.05 | 0.32 | −0.09 | 0.03 | −0.19 | 0.25 | −0.07 | 0.17 | 0.06 | 0.11 | 0.29 | 0.22 | 0.26 | 0.45 |
| Max | 0.39 | −0.10 | 0.34 | 0.18 | −0.05 | 0.23 | −0.14 | 0.15 | −0.25 | 0.15 | −0.24 | 0.26 | −0.10 | 0.02 | 0.18 | 0.22 | 0.25 | 0.35 |
| MDM2-hmut | 0.61 | 0.26 | 0.40 | 0.31 | 0.36 | 0.41 | −0.22 | 0.37 | −0.48 | 0.43 | 0.01 | 0.55 | 0.17 | −0.05 | 0.33 | 0.32 | 0.22 | 0.50 |
| MDM2- | 0.16 | 0.08 | 0.28 | 0.55 | 0.01 | 0.11 | −0.15 | 0.07 | −0.17 | 0.27 | 0.05 | 0.16 | 0.16 | 0.03 | 0.22 | −0.03 | −0.14 | 0.44 |
| mWT | | | | | | | | | | | | | | | | | | |
| TERT-WT | −0.06 | −0.08 | −0.13 | −0.05 | −0.13 | 0.13 | 0.28 | −0.21 | 0.00 | 0.00 | −0.18 | −0.22 | −0.06 | 0.11 | 0.00 | −0.37 | −0.14 | 0.05 |
| TERT-DN | 0.50 | 0.30 | 0.39 | 0.30 | 0.29 | 0.32 | −0.09 | 0.38 | −0.49 | −0.02 | −0.29 | 0.58 | −0.19 | −0.41 | 0.18 | 0.04 | 0.50 | 0.45 |
| PTEN-WT | 0.44 | −0.06 | 0.22 | −0.22 | 0.27 | 0.38 | −0.22 | −0.03 | −0.21 | 0.02 | −0.21 | 0.23 | −0.08 | −0.34 | 0.26 | 0.06 | 0.22 | 0.42 |
| PTEN-A3 | 0.32 | −0.06 | 0.26 | −0.08 | −0.22 | 0.24 | −0.07 | −0.12 | −0.42 | −0.07 | −0.37 | 0.15 | −0.17 | −0.12 | 0.07 | 0.13 | 0.22 | 0.02 |
| PTEN- | 0.45 | −0.08 | 0.29 | 0.11 | 0.16 | 0.24 | −0.09 | 0.14 | −0.17 | −0.05 | −0.22 | 0.37 | −0.01 | −0.34 | 0.40 | 0.01 | 0.28 | 0.78 |
| G129R | | | | | | | | | | | | | | | | | | |
| Bcl-2 | 0.21 | 0.14 | 0.05 | 0.18 | −0.01 | 0.24 | −0.11 | −0.01 | −0.32 | −0.04 | 0.23 | 0.01 | 0.26 | 0.04 | 0.11 | 0.25 | 0.09 | |
| per2 | 0.23 | 0.15 | 0.21 | 0.18 | 0.24 | 0.42 | −0.07 | 0.14 | −0.18 | 0.02 | 0.22 | 0.12 | −0.09 | 0.34 | 0.16 | 0.44 | 0.21 | |
| per3 | 0.13 | −0.02 | 0.20 | 0.11 | 0.18 | 0.41 | −0.07 | 0.02 | 0.03 | −0.23 | 0.16 | 0.03 | 0.24 | −0.06 | 0.40 | 0.13 | 0.25 | 0.18 |
| Cyclin | −0.25 | −0.35 | 0.01 | −0.24 | −0.13 | −0.07 | 0.05 | 0.00 | 0.25 | −0.38 | 0.11 | −0.20 | 0.13 | −0.16 | 0.05 | 0.00 | 0.07 | 0.04 |
| D1-11 | | | | | | | | | | | | | | | | | | |
| STAT2-4 | −0.18 | −0.17 | −0.08 | −0.08 | −0.20 | 0.15 | −0.52 | −0.36 | 0.30 | −0.18 | 0.20 | −0.26 | 0.12 | 0.43 | 0.26 | 0.10 | −0.35 | 0.04 |
| TSC1-4 | 0.25 | 0.30 | −0.19 | 0.07 | 0.23 | 0.39 | 0.31 | 0.16 | −0.27 | 0.26 | 0.13 | 0.39 | 0.23 | 0.31 | 0.09 | 0.18 | 0.22 | 0.02 |
| Bad-22 | 0.15 | 0.07 | 0.25 | 0.01 | 0.50 | 0.36 | 0.11 | 0.37 | 0.04 | −0.02 | 0.07 | 0.30 | 0.27 | 0.16 | 0.05 | 0.20 | 0.30 | 0.22 |
| FAPP-4 | −0.54 | −0.21 | −0.28 | 0.03 | −0.12 | −0.18 | 0.05 | 0.03 | 0.39 | −0.20 | 0.26 | −0.26 | 0.04 | 0.40 | −0.09 | −0.17 | −0.04 | −0.11 |
| FHO-6 | 0.13 | 0.01 | −0.01 | −0.21 | 0.26 | 0.39 | −0.17 | 0.03 | −0.01 | −0.02 | 0.03 | 0.00 | 0.29 | 0.12 | 0.27 | 0.22 | 0.14 | 0.18 |
| F25 + | 0.34 | 0.38 | 0.37 | 0.05 | 0.25 | 0.45 | −0.23 | 0.10 | −0.20 | 0.10 | 0.14 | 0.41 | 0.13 | 0.08 | 0.10 | 0.30 | 0.28 | 0.28 |
| lactacystin | | | | | | | | | | | | | | | | | | |
| F25 + | 0.01 | 0.17 | −0.07 | −0.06 | −0.02 | −0.14 | −0.17 | −0.11 | 0.18 | 0.09 | 0.31 | 0.17 | 0.02 | 0.34 | 0.02 | 0.09 | −0.17 | −0.09 |
| ONO5046 | | | | | | | | | | | | | | | | | | |
| F25 + | −0.09 | −0.17 | −0.08 | −0.06 | −0.02 | 0.02 | −0.30 | −0.05 | 0.25 | 0.10 | 0.34 | −0.11 | 0.20 | 0.42 | −0.08 | 0.22 | −0.19 | −0.08 |
| CA-074 | | | | | | | | | | | | | | | | | | |
| F25 + PQQ | −0.23 | −0.36 | −0.21 | −0.21 | −0.14 | −0.08 | −0.24 | −0.14 | 0.30 | 0.05 | 0.24 | −0.31 | 0.13 | 0.42 | −0.17 | 0.18 | −0.38 | −0.12 |
| F25 + | 0.16 | 0.02 | 0.37 | 0.19 | −0.04 | 0.06 | −0.39 | −0.07 | −0.10 | −0.10 | −0.03 | 0.02 | −0.17 | 0.00 | 0.34 | −0.07 | −0.14 | 0.31 |
| PD98059 | | | | | | | | | | | | | | | | | | |
| F25 + ALLN | 0.32 | 0.41 | 0.26 | 0.08 | 0.16 | 0.22 | −0.17 | −0.09 | −0.31 | 0.18 | −0.21 | 0.29 | −0.08 | −0.07 | −0.04 | −0.15 | 0.09 | 0.04 |
| F25 + | 0.47 | 0.28 | 0.19 | 0.05 | 0.26 | 0.52 | −0.41 | 0.17 | −0.40 | 0.41 | 0.15 | 0.39 | 0.22 | 0.10 | 0.12 | 0.45 | 0.20 | 0.24 |
| ONO3403 | | | | | | | | | | | | | | | | | | |
| F25 + | 0.40 | 0.17 | 0.30 | 0.39 | 0.28 | 0.25 | −0.37 | 0.36 | −0.08 | 0.29 | 0.23 | 0.30 | 0.32 | −0.02 | 0.30 | 0.40 | 0.16 | 0.54 |
| Y27632 | | | | | | | | | | | | | | | | | | |

TABLE 11

| | | | | C/ | C/ | per- | p33/ | | CD44/ | CD44/ | CD44/ | Jagged- | p94- | p94- | E2F- |
|---|---|---|---|---|---|---|---|---|---|---|---|---|---|---|---|
| | HNF3b | HNF4 | coup | EBPa | EBPb | 1 | ING1 | T.Tn | H-CAM | 3E | 3s | 1 | WT | mut | High |
| HNF3b | 1.00 | | | | | | | | | | | | | | |
| HNF4 | 0.06 | 1.00 | | | | | | | | | | | | | |
| coup | 0.01 | 0.17 | 1.00 | | | | | | | | | | | | |
| C/EBPa | 0.19 | −0.11 | 0.20 | 1.00 | | | | | | | | | | | |
| C/EBPb | 0.10 | −0.02 | 0.34 | 0.40 | 1.00 | | | | | | | | | | |
| per-1 | 0.47 | 0.11 | −0.25 | 0.01 | −0.06 | 1.00 | | | | | | | | | |
| p33/ING1 | −0.29 | −0.11 | 0.32 | 0.25 | 0.31 | −0.31 | 1.00 | | | | | | | | |
| T.Tn | 0.02 | 0.17 | −0.12 | 0.16 | −0.04 | −0.01 | −0.10 | 1.00 | | | | | | | |
| CD44/H- | −0.23 | 0.06 | 0.10 | −0.06 | 0.09 | −0.11 | 0.45 | −0.11 | 1.00 | | | | | | |
| CAM | | | | | | | | | | | | | | | |
| CD44/3E | −0.24 | −0.11 | −0.27 | 0.02 | 0.02 | −0.10 | 0.16 | 0.10 | 0.18 | 1.00 | | | | | |
| CD44/3s | −0.01 | −0.05 | −0.10 | 0.08 | 0.11 | −0.16 | 0.31 | 0.01 | 0.19 | 0.67 | 1.00 | | | | |
| Jagged-1 | −0.11 | −0.02 | 0.09 | 0.04 | −0.28 | −0.12 | −0.01 | 0.01 | −0.01 | 0.17 | 0.08 | 1.00 | | | |
| p94-WT | 0.04 | 0.02 | 0.39 | −0.01 | 0.22 | −0.16 | 0.23 | −0.27 | −0.11 | 0.04 | 0.14 | 0.04 | 1.00 | | |
| p94-mut | 0.04 | 0.06 | 0.07 | −0.26 | 0.06 | 0.02 | −0.06 | −0.24 | 0.00 | −0.22 | −0.20 | −0.20 | 0.62 | 1.00 | |
| E2F-High | 0.25 | −0.02 | 0.12 | −0.15 | 0.10 | 0.14 | −0.06 | −0.10 | −0.10 | 0.15 | 0.28 | −0.13 | 0.39 | 0.36 | 1.00 |

TABLE 12

| | HNF3b | HNF4 | coup | C/EBPa | C/EBPb | per-1 | p33/ING1 | T.Tn |
|---|---|---|---|---|---|---|---|---|
| E2F-Low | 0.24 | 0.10 | 0.11 | −0.09 | −0.04 | −0.14 | −0.12 | 0.09 |
| FBRCA1-13 | 0.04 | 0.10 | −0.21 | 0.02 | −0.09 | 0.06 | 0.03 | −0.07 |
| FMDM2hwt-6 | 0.11 | 0.03 | −0.43 | −0.17 | −0.47 | 0.27 | −0.27 | −0.01 |
| p27-3 | 0.13 | 0.29 | 0.52 | −0.03 | 0.05 | −0.27 | 0.14 | −0.10 |
| CAPN10-10 | −0.06 | 0.02 | 0.47 | 0.32 | 0.46 | −0.18 | 0.27 | 0.09 |
| c-myc-1 | −0.01 | 0.16 | 0.46 | −0.08 | 0.24 | 0.07 | −0.03 | −0.07 |
| MSSP-10 | 0.02 | −0.12 | −0.02 | −0.23 | −0.39 | −0.18 | −0.12 | −0.10 |
| MM1 | −0.13 | −0.01 | 0.42 | 0.23 | 0.45 | −0.06 | 0.28 | −0.23 |
| AMY1 | 0.07 | 0.06 | 0.47 | 0.28 | 0.22 | −0.28 | 0.37 | 0.01 |
| Max | 0.07 | −0.08 | 0.30 | 0.34 | 0.24 | −0.24 | 0.34 | 0.08 |
| MDM2-hmut | 0.18 | 0.04 | 0.61 | 0.45 | 0.56 | 0.08 | 0.26 | −0.12 |
| MDM2-mWT | 0.26 | −0.06 | 0.34 | 0.11 | 0.23 | 0.15 | −0.22 | 0.02 |
| TERT-WT | 0.11 | −0.07 | −0.06 | −0.19 | −0.01 | −0.04 | −0.15 | 0.01 |
| TERT-DN | −0.17 | −0.16 | 0.22 | 0.55 | 0.62 | −0.04 | 0.36 | 0.01 |
| PTEN-WT | −0.21 | −0.10 | 0.23 | 0.08 | 0.29 | −0.29 | 0.18 | 0.14 |
| PTEN-A3 | −0.09 | −0.10 | −0.02 | 0.04 | −0.06 | −0.05 | 0.23 | −0.10 |
| PTEN-G129R | 0.04 | 0.01 | 0.21 | 0.25 | 0.27 | −0.11 | 0.22 | 0.19 |
| Bcl-2 | 0.09 | −0.02 | 0.28 | 0.14 | 0.24 | −0.02 | 0.22 | −0.05 |
| per2 | −0.27 | 0.02 | 0.32 | 0.16 | 0.14 | −0.07 | 0.36 | 0.03 |
| per3 | −0.23 | 0.12 | 0.15 | 0.08 | −0.18 | −0.02 | 0.18 | 0.18 |
| Cyclin D1-11 | −0.13 | −0.02 | −0.34 | −0.02 | −0.30 | −0.13 | 0.15 | 0.16 |
| STAT2-4 | −0.14 | 0.23 | 0.10 | −0.30 | −0.29 | −0.17 | −0.10 | −0.05 |
| TSC1-4 | 0.20 | 0.03 | 0.29 | −0.05 | 0.04 | 0.14 | 0.17 | −0.30 |
| Bad-22 | 0.22 | −0.03 | 0.19 | 0.24 | 0.16 | 0.11 | 0.33 | 0.10 |
| FAPP-4 | 0.07 | 0.05 | −0.02 | −0.28 | −0.18 | −0.12 | −0.16 | 0.06 |
| FHO-6 | 0.05 | 0.03 | 0.32 | −0.11 | −0.01 | 0.14 | 0.17 | −0.04 |
| F25 + lactacystin | 0.06 | 0.10 | 0.39 | 0.19 | 0.05 | 0.02 | 0.15 | −0.12 |
| F25 + ONO5046 | 0.18 | 0.00 | 0.21 | 0.12 | −0.09 | −0.05 | 0.00 | −0.08 |
| F25 + CA-074 | −0.06 | 0.10 | 0.30 | −0.02 | −0.26 | −0.30 | 0.04 | −0.08 |
| F25 + PQQ | −0.02 | 0.11 | 0.21 | −0.28 | −0.35 | −0.24 | −0.01 | 0.05 |
| F25 + PD98059 | −0.07 | −0.01 | 0.26 | 0.12 | 0.16 | −0.16 | −0.04 | 0.04 |
| F25 + ALLN | 0.11 | 0.13 | 0.19 | 0.22 | 0.30 | 0.20 | −0.06 | −0.16 |
| F25 + ONO3403 | 0.17 | 0.12 | 0.47 | 0.17 | 0.22 | 0.04 | 0.16 | −0.30 |
| F25 + Y27632 | 0.20 | 0.30 | 0.45 | 0.25 | 0.16 | 0.16 | 0.10 | −0.06 |

| | CD44/H-CAM | CD44/3E | CD44/3s | Jagged-1 | p94-WT | p94-mut | E2F-High |
|---|---|---|---|---|---|---|---|
| E2F-Low | −0.05 | 0.14 | 0.31 | −0.03 | 0.06 | −0.09 | 0.57 |
| FBRCA1-13 | 0.03 | 0.22 | 0.22 | 0.12 | 0.02 | −0.13 | −0.12 |
| FMDM2hwt-6 | −0.11 | 0.17 | 0.01 | 0.47 | −0.05 | −0.06 | −0.03 |
| p27-3 | 0.16 | 0.05 | 0.19 | 0.33 | 0.39 | −0.04 | 0.01 |
| CAPN10-10 | 0.12 | −0.23 | −0.19 | −0.24 | 0.26 | 0.32 | 0.13 |
| c-myc-1 | 0.21 | −0.25 | −0.29 | 0.03 | 0.31 | 0.31 | 0.23 |
| MSSP-10 | −0.03 | 0.09 | 0.13 | 0.38 | 0.09 | −0.08 | 0.17 |
| MM1 | 0.31 | −0.03 | 0.05 | −0.12 | 0.16 | 0.00 | 0.16 |
| AMY1 | 0.06 | −0.23 | −0.03 | −0.20 | 0.10 | −0.01 | 0.00 |
| Max | 0.11 | 0.01 | 0.18 | −0.09 | −0.07 | −0.23 | −0.03 |
| MDM2-hmut | 0.12 | −0.12 | 0.12 | −0.11 | 0.29 | 0.07 | 0.25 |
| MDM2-mWT | −0.18 | −0.14 | −0.16 | −0.15 | −0.09 | 0.05 | 0.35 |
| TERT-WT | −0.15 | −0.17 | 0.08 | −0.34 | −0.09 | 0.17 | 0.09 |
| TERT-DN | 0.09 | 0.13 | 0.12 | −0.35 | 0.01 | −0.01 | 0.14 |
| PTEN-WT | −0.06 | −0.04 | 0.00 | −0.33 | 0.12 | 0.03 | 0.06 |
| PTEN-A3 | −0.08 | −0.30 | −0.32 | −0.33 | 0.04 | 0.14 | −0.16 |
| PTEN-G129R | −0.17 | −0.11 | 0.03 | −0.35 | 0.05 | −0.01 | 0.10 |
| Bcl-2 | 0.14 | −0.05 | 0.08 | −0.15 | −0.08 | 0.03 | −0.01 |
| per2 | 0.28 | −0.03 | −0.13 | −0.24 | 0.11 | 0.16 | −0.03 |
| per3 | 0.14 | −0.01 | −0.02 | 0.02 | 0.15 | 0.10 | −0.08 |
| Cyclin D1-11 | 0.00 | 0.37 | 0.20 | 0.10 | −0.15 | −0.14 | 0.06 |
| STAT2-4 | 0.01 | −0.17 | −0.39 | 0.41 | 0.01 | 0.08 | −0.12 |
| TSC1-4 | 0.28 | −0.19 | 0.13 | −0.09 | 0.25 | 0.02 | 0.10 |
| Bad-22 | 0.20 | −0.04 | 0.33 | 0.03 | 0.22 | −0.07 | 0.22 |
| FAPP-4 | 0.05 | −0.07 | −0.05 | 0.01 | −0.13 | 0.04 | 0.25 |
| FHO-6 | 0.01 | −0.16 | −0.23 | 0.01 | 0.43 | 0.24 | 0.04 |
| F25 + lactacystin | 0.01 | 0.09 | 0.04 | 0.16 | 0.44 | 0.07 | 0.09 |
| F25 + ONO5046 | −0.01 | −0.04 | 0.06 | 0.42 | 0.20 | −0.01 | −0.01 |
| F25 + CA-074 | 0.21 | −0.03 | 0.20 | 0.49 | 0.29 | −0.03 | 0.00 |
| F25 + PQQ | 0.09 | −0.13 | 0.01 | 0.53 | 0.20 | −0.06 | −0.18 |
| F25 + PD98059 | −0.03 | −0.26 | −0.36 | −0.01 | 0.21 | 0.43 | 0.08 |
| F25 + ALLN | 0.00 | −0.37 | −0.50 | −0.15 | 0.32 | 0.33 | −0.08 |
| F25 + ONO3403 | 0.06 | 0.02 | 0.15 | −0.02 | 0.48 | 0.13 | 0.22 |
| F25 + Y27632 | 0.01 | 0.00 | −0.09 | −0.06 | 0.25 | 0.03 | 0.20 |

TABLE 13

| | E2F-Low | FBRCA1-13 | FMDM2hwt-6 | p27-3 | CAPN10-10 | c-myc-1 | MSSP-10 | MM1 |
|---|---|---|---|---|---|---|---|---|
| E2F-Low | 1.00 | | | | | | | |
| FBRCA1-13 | 0.08 | 1.00 | | | | | | |
| FMDM2hwt-6 | −0.02 | 0.48 | 1.00 | | | | | |
| p27-3 | 0.26 | 0.22 | −0.04 | 1.00 | | | | |
| CAPN10-10 | 0.08 | −0.57 | −0.70 | 0.13 | 1.00 | | | |
| c-myc-1 | 0.05 | −0.37 | −0.38 | 0.36 | 0.60 | 1.00 | | |
| MSSP-10 | 0.38 | 0.40 | 0.59 | 0.29 | −0.47 | −0.19 | 1.00 | |
| MM1 | 0.04 | 0.00 | −0.38 | 0.23 | 0.32 | 0.42 | −0.08 | 1.00 |
| AMY1 | 0.21 | −0.16 | −0.57 | 0.21 | 0.49 | 0.16 | −0.32 | 0.29 |
| Max | 0.14 | 0.03 | −0.47 | 0.16 | 0.33 | 0.13 | −0.23 | 0.25 |
| MDM2-hmut | 0.19 | −0.19 | −0.51 | 0.30 | 0.50 | 0.40 | −0.31 | 0.47 |
| MDM2-mWT | 0.44 | −0.14 | −0.31 | −0.05 | 0.25 | 0.34 | −0.09 | 0.29 |
| TERT-WT | 0.02 | 0.10 | −0.17 | −0.26 | −0.04 | 0.04 | −0.22 | 0.01 |
| TERT-DN | −0.03 | −0.10 | −0.60 | −0.15 | 0.51 | 0.18 | −0.49 | 0.53 |
| PTEN-WT | 0.10 | −0.04 | −0.46 | 0.11 | 0.33 | 0.18 | −0.18 | 0.37 |
| PTEN-A3 | −0.24 | −0.07 | −0.39 | −0.06 | 0.20 | 0.04 | −0.21 | 0.01 |
| PTEN-G129R | 0.39 | 0.04 | −0.48 | 0.21 | 0.55 | 0.21 | −0.17 | 0.30 |
| Bcl-2 | −0.14 | −0.08 | −0.39 | 0.02 | 0.09 | 0.21 | −0.26 | 0.19 |
| per2 | −0.19 | −0.44 | −0.56 | −0.01 | 0.50 | 0.36 | −0.48 | 0.24 |
| per3 | −0.16 | −0.28 | −0.20 | 0.04 | 0.23 | 0.21 | −0.26 | 0.10 |
| Cyclin D1-11 | 0.21 | 0.32 | 0.43 | −0.18 | −0.36 | −0.55 | 0.30 | −0.28 |
| STAT2-4 | −0.10 | −0.15 | 0.23 | 0.16 | −0.07 | 0.23 | 0.13 | −0.20 |
| TSC1-4 | −0.09 | 0.04 | −0.29 | 0.40 | 0.03 | 0.34 | 0.02 | 0.43 |
| Bad-22 | 0.04 | −0.21 | −0.13 | 0.16 | 0.27 | 0.19 | −0.08 | 0.24 |
| FAPP-4 | 0.34 | −0.22 | 0.08 | 0.01 | −0.05 | −0.03 | 0.28 | −0.05 |
| FHO-6 | −0.12 | 0.00 | 0.09 | 0.25 | 0.02 | 0.14 | 0.08 | 0.06 |
| F25 + lactacystin | −0.17 | −0.09 | −0.11 | 0.28 | 0.13 | 0.27 | −0.07 | 0.39 |
| F25 + ONO5046 | −0.07 | −0.07 | 0.14 | 0.37 | −0.06 | 0.15 | 0.26 | −0.01 |
| F25 + CA-074 | 0.14 | −0.01 | 0.20 | 0.43 | −0.12 | 0.12 | 0.35 | 0.14 |
| F25 + PQQ | 0.03 | 0.10 | 0.40 | 0.52 | −0.28 | 0.08 | 0.50 | −0.03 |
| F25 + PD98059 | 0.02 | −0.56 | −0.45 | −0.08 | 0.61 | 0.45 | −0.44 | 0.05 |
| F25 + ALLN | −0.37 | −0.07 | −0.27 | −0.04 | 0.29 | 0.40 | −0.44 | 0.23 |
| F25 + ONO3403 | −0.09 | 0.06 | −0.22 | 0.40 | 0.11 | 0.31 | −0.05 | 0.29 |
| F25 + Y27632 | 0.23 | −0.04 | −0.17 | 0.36 | 0.42 | 0.34 | −0.11 | 0.10 |
| | AMY1 | Max | MDM2-hmut | MDM2-mWT | TERT-WT | TERT-DN | PTEN-WT | PTEN-A3 |
| E2F-Low | | | | | | | | |
| FBRCA1-13 | | | | | | | | |
| FMDM2hwt-6 | | | | | | | | |
| p27-3 | | | | | | | | |
| CAPN10-10 | | | | | | | | |
| c-myc-1 | | | | | | | | |
| MSSP-10 | | | | | | | | |
| MM1 | | | | | | | | |
| AMY1 | 1.00 | | | | | | | |
| Max | 0.56 | 1.00 | | | | | | |
| MDM2-hmut | 0.54 | 0.46 | 1.00 | | | | | |
| MDM2-mWT | 0.36 | 0.16 | 0.52 | 1.00 | | | | |
| TERT-WT | 0.30 | 0.37 | 0.02 | 0.32 | 1.00 | | | |
| TERT-DN | 0.28 | 0.48 | 0.43 | 0.22 | 0.06 | 1.00 | | |
| PTEN-WT | 0.46 | 0.45 | 0.27 | 0.00 | 0.27 | 0.44 | 1.00 | |
| PTEN-A3 | 0.29 | 0.42 | 0.11 | −0.14 | −0.06 | 0.18 | 0.16 | 1.00 |
| PTEN-G129R | 0.48 | 0.48 | 0.43 | 0.36 | 0.09 | 0.54 | 0.55 | 0.18 |
| Bcl-2 | 0.37 | 0.64 | 0.45 | 0.30 | 0.38 | 0.31 | 0.16 | 0.40 |
| per2 | 0.49 | 0.37 | 0.29 | −0.08 | −0.10 | 0.42 | 0.32 | 0.36 |
| per3 | 0.30 | 0.16 | 0.12 | −0.19 | −0.14 | 0.05 | 0.18 | 0.14 |
| Cyclin D1-11 | −0.25 | 0.19 | −0.34 | −0.25 | −0.02 | −0.06 | −0.09 | −0.07 |
| STAT2-4 | 0.03 | −0.02 | −0.28 | −0.14 | 0.06 | −0.32 | −0.09 | −0.10 |
| TSC1-4 | 0.21 | 0.15 | 0.31 | −0.06 | 0.01 | 0.05 | 0.18 | 0.29 |
| Bad-22 | 0.04 | −0.01 | 0.28 | −0.22 | −0.21 | 0.16 | 0.06 | −0.13 |
| FAPP-4 | 0.10 | −0.22 | −0.31 | 0.13 | 0.07 | −0.16 | −0.11 | −0.31 |
| FHO-6 | 0.13 | −0.02 | 0.13 | −0.15 | −0.17 | −0.12 | 0.26 | 0.16 |
| F25 + lactacystin | 0.21 | 0.08 | 0.33 | 0.11 | −0.06 | 0.14 | 0.13 | −0.02 |
| F25 + ONO5046 | −0.18 | −0.05 | 0.08 | −0.10 | −0.24 | −0.28 | −0.28 | −0.16 |
| F25 + CA-074 | 0.02 | 0.00 | 0.06 | −0.15 | −0.11 | −0.41 | −0.12 | −0.24 |
| F25 + PQQ | −0.13 | −0.06 | −0.11 | −0.24 | −0.16 | −0.62 | −0.16 | −0.15 |
| F25 + PD98059 | 0.21 | 0.09 | 0.19 | 0.28 | 0.06 | 0.29 | 0.03 | 0.06 |
| F25 + ALLN | 0.03 | −0.08 | 0.14 | −0.02 | 0.01 | 0.27 | 0.12 | 0.29 |
| F25 + ONO3403 | 0.28 | 0.20 | 0.55 | 0.11 | 0.00 | 0.08 | 0.20 | 0.32 |
| F25 + Y27632 | 0.27 | 0.19 | 0.47 | 0.25 | −0.25 | 0.13 | −0.05 | 0.12 |

TABLE 14

| | PTEN-G129R | Bcl-2 | per2 | per3 | Cyclin D1- | STAT2-4 | TSC1-4 | Bad-22 | FAPP-4 |
|---|---|---|---|---|---|---|---|---|---|
| PTEN-G129R | 1.00 | | | | | | | | |
| Bcl-2 | 0.04 | 1.00 | | | | | | | |
| per2 | 0.19 | 0.34 | 1.00 | | | | | | |
| per3 | 0.10 | 0.05 | 0.78 | 1.00 | | | | | |
| Cyclin D1-11 | 0.04 | −0.13 | −0.17 | 0.04 | 1.00 | | | | |
| STAT2-4 | −0.28 | 0.05 | 0.13 | 0.30 | 0.16 | 1.00 | | | |
| TSC1-4 | 0.00 | 0.32 | 0.32 | 0.30 | −0.42 | 0.01 | 1.00 | | |
| Bad-22 | 0.18 | −0.19 | 0.16 | 0.27 | −0.13 | −0.16 | 0.37 | 1.00 | |
| FAPP-4 | −0.11 | −0.16 | −0.01 | −0.09 | 0.02 | 0.28 | −0.03 | −0.01 | 1.00 |
| FHO-6 | 0.03 | −0.06 | 0.33 | 0.45 | 0.03 | 0.25 | 0.35 | 0.24 | 0.11 |
| F25 + lactacystin | 0.09 | 0.13 | 0.25 | 0.35 | −0.13 | 0.15 | 0.37 | 0.32 | −0.05 |
| F25 + ONO5046 | −0.29 | 0.14 | −0.15 | −0.01 | −0.05 | 0.30 | 0.26 | 0.12 | 0.03 |
| F25 + CA-074 | −0.34 | −0.01 | −0.04 | 0.23 | 0.06 | 0.43 | 0.24 | 0.21 | 0.12 |
| F25 + PQQ | −0.30 | −0.07 | −0.20 | 0.09 | 0.11 | 0.44 | 0.14 | 0.08 | 0.12 |
| F25 + PD98059 | 0.15 | 0.02 | 0.22 | 0.06 | −0.21 | 0.15 | −0.23 | 0.01 | −0.02 |
| F25 + ALLN | −0.09 | 0.05 | 0.24 | 0.04 | −0.55 | −0.02 | 0.28 | 0.03 | −0.22 |
| F25 + ONO3403 | 0.03 | 0.35 | 0.17 | 0.14 | −0.25 | 0.05 | 0.57 | 0.18 | −0.32 |
| F25 + Y27632 | 0.35 | 0.08 | 0.28 | 0.23 | −0.01 | 0.04 | 0.07 | 0.12 | −0.29 |

| | FHO-6 | +lactacystin | +ONO5046; | +CA-074 | F25 + PQQ | PD98059 | F25 + ALLN | + ONO3403 | +Y27632 |
|---|---|---|---|---|---|---|---|---|---|
| PTEN-G129R | | | | | | | | | |
| Bcl-2 | | | | | | | | | |
| per2 | | | | | | | | | |
| per3 | | | | | | | | | |
| Cyclin D1-11 | | | | | | | | | |
| STAT2-4 | | | | | | | | | |
| TSC1-4 | | | | | | | | | |
| Bad-22 | | | | | | | | | |
| FAPP-4 | | | | | | | | | |
| FHO-6 | 1.00 | | | | | | | | |
| F25 + lactacystin | 0.46 | 1.00 | | | | | | | |
| F25 + ONO5046 | 0.12 | 0.22 | 1.00 | | | | | | |
| F25 + CA-074 | 0.20 | 0.24 | 0.62 | 1.00 | | | | | |
| F25 + PQQ | 0.30 | 0.10 | 0.56 | 0.79 | 1.00 | | | | |
| F25 + PD98059 | −0.13 | 0.04 | 0.13 | 0.08 | −0.17 | 1.00 | | | |
| F25 + ALLN | 0.16 | 0.28 | 0.00 | −0.13 | −0.27 | 0.34 | 1.00 | | |
| F25 + ONO3403 | 0.33 | 0.58 | 0.17 | 0.24 | 0.10 | −0.07 | 0.29 | 1 | |
| F25 + Y27632 | 0.10 | 0.25 | 0.03 | 0.04 | 0.00 | 0.06 | 0.04 | 0.46 | 1 |

From the result of the Tables 3 to 14, there were identified 71 pairs of genes showing positive correlations (r>0.5) and 17 pairs of genes showing negative correlations (r<-0.5). A part of those gene combinations and functional relationship thereof is shown in Table 15.

TABLE 15

r values

1. Combinations of genes belonging to the same family
where correlation is naturally expected

| | | | |
|---|---|---|---|
| Ha-ras | v.s. | Ki-ras | 0.71 |
| Ha-ras | v.s. | N-ras | 0.56 |
| Ki-ras | v.s. | N-ras | 0.56 |
| v-src | v.s. | erbB2 | 0.66 |
| cystatin α | v.s. | cystatin E | 0.54 |
| PKCα-KN | v.s. | PKCε-KN | 0.68 |
| ERK-DN | v.s. | p38-DN | 0.63 |
| JNK-DN | v.s. | p38-DN | 0.54 |
| caspase-1 | v.s. | caspase-3 | 0.49 |
| Hsdj | v.s. | DnaJ-61 | 0.40 |

2. Combination of a transcription factor and the target gene

| | | | |
|---|---|---|---|
| p53 | v.s. | p21 | 0.56 |

3. Presence of a product at the downstream of the signal of
another product

| | | | |
|---|---|---|---|
| Ha-las | -> | RhoA-DN | −0.55 |
| N-ras | -> | ERK-DN | −0.51 |
| Ras-DN | -> | Akt-DN | 0.40 |
| IKK-DN | -> | IkB-SR | 0.75 |
| PDGFR-Δ | -> | ERK-DN | 0.62 |
| CaMKIIα-active | -> | coup | 0.56 |
| p53 | -> | caspase-3 | 0.52 |
| IkB-SR | -> | p53 | 0.52 |
| IKK-DN | -> | p21 | 0.62 |
| glucocorticoid-R | -> | STAT1 | 0.78 |

4. Protease and its substrate

| | | | |
|---|---|---|---|
| m-calpain | -> | PDGFR-Δ | 0.54 |

5. Simultaneous expression

| | | | |
|---|---|---|---|
| p16 | v.s. | Bax | 0.56 |
| hsp90 | v.s. | caspase-3 | 0.63 |
| JNK-DN | v.s. | μ-calpain | 0.48 |

6. Binding protein

| | | | |
|---|---|---|---|
| p53 | v.s. | MDM2-WT | −0.56 |
| p53 | v.s. | MDM2-mutated | 0.62 |
| hsp90 | v.s. | TERT-DN | 0.53 |

As a result of the above method, there was constructed a gene function database where correlation among the genes having known functions was made clear. By referring to the database, it is now possible to easily elucidate the functions of the gene having unknown functions.

### INDUSTRIAL APPLICABILITY

As mentioned in details hereinabove, the invention of this application provides a novel method of analysis of functions of function-unknown genes useful as a genetic material for the Pharmacogenomics and for the manufacture of various useful proteins by means of genetic engineering and also provides a gene function database to be used for the analysis as well as a method for constructing the database.

1. A method for construction of a gene function database, which comprises:
(a) measuring the viabilities, against a plural number of drugs (D₁, D₂, D₃, . . . Dₙ) at various concentrations, of transformed eukaryotic cells overexpressing a plural number of function-known genes (g₁, g₂, g₃, . . . gₙ) and their parental cell lines;
(b) calculating the ratio of the concentration value of the drug to inhibit the viability of the transformed cell to an extent of 40% (IC₄₀ value) to the IC₄₀ value of the parental cell line;
(c) calculating the logarithmic values of the ratios of the above (b) for the known genes (g₁, g₂, g₃, . . . gₙ); and
(d) calculating the correlation coefficient among the known genes (g₁, g₂, g₃, . . . gₙ) for the logarithmic values of the above (c).

2. The method for construction of the gene function database according to claim 1, wherein “n” of the function-known genes (gₙ) is 50 or more.

3. The method for construction of the gene function database according to claim 1, wherein “n” of the drugs (Dₙ) is 40 or more.

4. A gene function database, which is constructed by any method of claims 1 to 3.

5. A method of analyzing functions of a function-unknown gene (gₓ) on the basis of DSPA (Drug Sensitivity Pattern Analysis) using the gene function database of claim 4, which comprises:
(i) measuring the IC₄₀ value for each drug from the viabilities at various concentrations of a plural number of drugs (D₁, D₂, D₃, . . . Dₙ) for transformed eukaryotic cells overexpressing the unknown gene (gₓ),
(ii) calculating the correlation coefficients between the unknown gene (gₓ) and known genes (g₁, g₂, g₃, . . . gₙ) from the IC₄₀ values of the above (i) by the same method as in the calculation of the correlation coefficients among the known genes (g₁, g₂, g₃, . . . gₙ) of the gene function database, and
(iii) determining that the function of the known gene showing a significant correlation coefficient to the unknown gene (gₓ) is related to the function of the unknown gene (gₓ).

6. The method for constructing the database according to claim 1, wherein the data of the unknown gene (gₓ) whose function is determined by the method of claim 5 is added to the database as a data of the function-known gene.

7. A gene function database, which is constructed by the method of claim 6.
